(19) United States
(12) Patent Application Publication
(10) Pub. No.: US 2022/0354845 A1
(43) Pub. Date: Nov. 10, 2022

(54) METHODS FOR ENHANCING DNA DAMAGE AND APOPTOSIS OF LEUKEMIC CELLS

(71) Applicant: Thomas Jefferson University, Philadelphia, PA (US)

(72) Inventors: Bruno Calabretta, Philadelphia, PA (US); Alexander Mazo, Cherry Hill, NJ (US); Svetlana Petruk, Voorhees, NJ (US); Patrizia Porazzi, Philadelphia, PA (US)

(21) Appl. No.: 17/637,004

(22) PCT Filed: Aug. 21, 2020
(86) PCT No.: PCT/US20/47463

§ 371 Date: Feb. 21, 2022

Related U.S. Application Data
(60) Provisional application No. 62/890,329, filed on Aug. 22, 2019.

(51) Int. Cl. A61K 31/497; A61K 31/7068; A61P 35/02

(52) U.S. Cl. CPC A61K 31/497; A61K 31/7068; A61P 35/02

(57) ABSTRACT

The present invention relates to methods of treating cancer in a subject in need thereof by treatments which enhance DNA damage and apoptosis of leukemic cells.

### CROSS-REFERENCE TO RELATED APPLICATION

The present application is entitled to priority under 35 U.S.C. § 119(e) to U.S. Provisional Patent Application 62/890,329, filed Aug. 22, 2019, which is hereby incorporated by reference in its entirety.

### STATEMENT REGARDING FEDERALLY SPONSORED RESEARCH OR DEVELOPMENT

This invention was made with government support under HL127895 and GM075141 awarded by the National Institutes of Health. The government has certain rights in the invention.

### BACKGROUND OF THE INVENTION

Acute myeloid leukemia (AML) results from the transformation of early hematopoietic progenitor cells (HPCs), leading to differentiation arrest and clonal expansion. The characterization of the AML's molecular landscape has revealed a high degree of heterogeneity and genetic complexity. In particular, genetically distinct sub-clones coexist in the same patient, creating obstacles for the development of effective, molecularly-targeted therapies. Thus, the frontline therapy for most AML patients remains non-targeted intensive induction chemotherapy followed by bone marrow transplant and/or consolidation therapy. This aggressive regimen is generally offered only to otherwise “fit” patients (typically young adult and/or of high-performance status), and an effective approach for elderly or unfit patients has not yet been established.

Two key features of the mechanism of chemotherapy-induced DNA damage that may improve its efficiency at reduced doses have not yet been exploited. First, most conventional chemotherapeutic agents induce DNA damage and cell death by targeting tumor cells during DNA replication. Second, chromatin structure influences the effects of DNA damaging agents and the efficiency of the DNA damage response. For example, constitutive compacted heterochromatin is more resistant to formation of double-strand breaks (DSBs). By contrast, histone deacetylase (HDAC) inhibitors, which cause chromatin relaxation, increase sensitivity of cancer cells to ionizing radiation (IR). Moreover, chemical profiling of genomic DNA damage sites induced by cytotoxic drugs suggests that DNA damage induced by the widely used Topoisomerase II inhibitors Daunomycin and Etoposide is not random, but instead preferentially targets transcriptionally active, de-condensed regions of the genome marked by the modified histones H3K36me3 and H3K9me1. By contrast, H3K27me3-marked, repressed regions of the genome are relatively inaccessible to these DNA-damaging agents.

In euchromatin, arrays of nucleosomes at repressed genes are marked with H3K27me3 and represent the most compact chromatin in the genome. Given that H3K27me3-modified chromatin is more resistant to DNA-damaging agents, it can be potentially targeted to improve the efficiency of cytotoxic agents.

In the present disclosure, chromatin assembly arrays (CAA) were used to examine at single-cell resolution the deposition of H3K27me3 and the recruitment of H3K27me3 EZH2 HMT on nascent DNA of CD34+ AML blasts. EZH2 was observed to be present on nascent DNA of tested AML cell lines and AML blasts from patients with newly diagnosed disease. Moreover, nascent chromatin in AML cell lines and AML primary samples exhibits a highly “condensed” post-replicative structure characterized by the rapid and stable association of H3K27me3 with nascent DNA. These findings provided the opportunity to rationally combine cytotoxic drugs and small molecule inhibitors of histone-modifying enzymes with the goal of enhancing DNA damage and apoptosis of AML blast cells. Moreover, the DNA-damaging and apoptosis-inducing effects of the EZH2 inhibitor/chemotherapy combination may be further improved by enhancing the targeting of nascent chromatin through enrichment of S-phase AML cells after release from G1 arrest induced by pharmacological inhibition of the CDK4/6 cell cycle regulatory kinases.

Current anti-cancer therapies include chemotherapeutic agents which target cells during DNA replication, and induce DNA damage-dependent apoptosis. Moreover, DNA damage causes primary and secondary cancers and contributes to many of the side effects of cancer therapy. It is likely that a more condensed structure of nucleosomes protects DNA from damage. There remains a need for methods of treating cancers such as leukemia that act to augment existing DNA-damaging chemotherapies. The present invention addresses and satisfies this need.

### SUMMARY OF THE INVENTION

The present disclosure is based on the discovery that cell cycle and histone methyltransferase inhibitors can be combined to enhance the ability of chemotherapies to kill cancer cells, thereby improving cancer treatments. This combination approach modifies the histone/DNA complexes such that the DNA is more accessible to DNA-damaging chemotherapy drugs.

As such, in certain aspects, the invention includes a method of treating cancer in a subject, the method comprising administering to the subject an effective amount of a histone methyltransferase inhibitor, and an effective amount of a chemotherapy.

In another aspect, the invention includes a method of treating cancer in a subject comprising administering to the subject an effective amount of GSK126, an effective amount of Doxorubicin, and an effective amount of cytarabine.

In another aspect, the invention includes a method of treating cancer in a subject comprising administering to a sample from the subject, a) a plurality of combinations of histone methyltransferase inhibitors and chemotherapies, b) determining the optimal combination of histone methyltransferase inhibitors and chemotherapies, and c) treating the subject with the optimal combination of histone methyltransferase inhibitors and chemotherapies.

In various embodiments, the method further comprises administering to the subject an effective amount of a cell cycle inhibitor.

In various embodiments, the method further comprises administering to the subject an effective amount of one or more additional chemotherapies.

In certain embodiments, the one or more additional chemotherapies are selected from the group consisting of a platinum drug, a Topoisomerase II inhibitor drug, an anthracycline drug, an antimetabolite drug, bleomycin, cisplatin, carboplatin, oxaliplatin, nedaplatin, Doxorubicin, daunorubicin, epirubicin, mitoxantrone, cytarabine, mercaptopurine, fludarabine, hydroxycarbamide, and methotrexate.

In various embodiments, the cell cycle inhibitor is administered first, followed by the histone methyltransferase inhibitor, followed by the chemotherapy.

In various embodiments, the cell cycle inhibitor, histone methyltransferase inhibitor, and chemotherapy are administered concurrently.

In various embodiments, the cell cycle inhibitor is administered first, followed by concurrent administration of the histone methyltransferase inhibitor and chemotherapy.

In various embodiments, the cell cycle inhibitor is an inhibitor of any one or both of CDK4 and CDK6.

In various embodiments, the CDK4 and CDK6 inhibitor is selected from the group consisting of abemaciclib, palbociclib, and ribociclib.

In various embodiments, the histone methyltransferase inhibitor is an inhibitor of the methylation of H3K27.

In certain embodiments, the histone methyltransferase inhibitor is an inhibitor of enzyme Enhancer of Zeste Homolog 2 (EZH2).

In various embodiments, the EZH2 inhibitor is selected from the group consisting of tazemetostat, GSK126, 3-deazaneplanocin A, and GSK343.

In various embodiments, the chemotherapy is a DNA-damaging agent.

In certain embodiments, the DNA-damaging agent is selected from the group consisting of a platinum drug and a Topoisomerase II inhibitor drug.

In various embodiments, the platinum drug is selected from the group consisting of cisplatin, carboplatin, oxaliplatin, and nedaplatin.

In various embodiments, the Topoisomerase II inhibitor is an anthracycline drug selected from the group consisting of Doxorubicin, daunorubicin, epirubicin, and mitoxantrone.

In various embodiments, the Topoisomerase II inhibitor is etoposide.

In various embodiments, the chemotherapy is bleomycin.

In various embodiments , the chemotherapy is an antimetabolite.

In various embodiments, the antimetabolite is selected from the group consisting of cytarabine, mercaptopurine, fludarabine, hydroxycarbamide, and methotrexate.

In various embodiments, the subject is a human.

In various embodiments, the cancer is a hematologic malignancy.

In certain embodiments, the hematologic malignancy is selected from the group consisting of acute myelogenous leukemia, acute myeloblastic leukemia, acute myeloid leukemia, and acute nonlymphocytic leukemia, acute monocytic leukemia, acute monoblastic leukemia, acute megakaryocytic leukemia, acute erythroblastic leukemia, and chronic myelogenous leukemia-blast crisis.

In certain embodiments, the subject is pre-screened for an ASXL1 mutation, wherein when the subject carries the ASXL1 mutation, an alternative treatment is administered.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1B illustrate the re-CHiP and chromatin assembly assays (CAA). FIG. 1A is a schematic diagram of the CAA assay. DNA is labeled with EdU in vivo. EdU and conjugated biotin are shown as light and dark circles on nascent DNA. Examined proteins are shown as squares. Proximity ligation assay (PLA) signals are shown bound to anti-biotin and anti-query antibodies. Following PLA, cells can be immunostained with antibodies to biotin, brdU, and cell differentiation markers. FIG. 1B, is a schematic diagram of re-ChiP with brdU. DNA is labeled with brdU in vivo, chromatin is cross-linked, sonicated, and immunoprecipitated (IPed) first with Ab to tested protein, and then IPed with Ab to brdU.

FIGS. 2A-2B illustrate the accumulation of H3K27me3 on nascent DNA of G-CSF mobilized CD34+ HPCs. In FIG. 2A, DNA was labeled with EdU for 15 min and chased to 1, 2, and 4 h. Following conjugation with biotin, CAA was performed between nascent DNA (biotin) and H3K27me3. Lower panels show PLA signals only. FIG. 2B displays quantification of the results of CAA experiments.

FIGS. 3A-3D illustrate accumulation of H3K27me3 on nascent DNA. FIG. 3A shows THP-1 cells labeled with EdU and stained according to the CAA assay. FIG. 3B is a quantification of the results of the CAA experiments. FIG. 3C shows DNA from human CD34+ primary acute myeloid leukemia (AML) samples were labeled with EdU for 15 minutes and chased for the indicated time. Following conjugation with biotin, CAA was performed between nascent DNA (biotin) and H3K27me3. PLA, Edu (biotin), and DAPI. Lower panels show PLA signals only. FIG. 3D shows a similar study was performed using a human AML sample which had a lower percentage of CD34+ cells which had been cultured both in vitro and as a xenograft in mice.

FIGS. 4A-4B illustrate the ability of EZH2 inhibitor to affect H3K27me3 binding. FIG. 4A shows THP-1 or AML primary cells were incubated with the EZH2 inhibitor GSK126 for 12 hours, switching the chromatin conformation from “closed” to “open”. Cells were labeled with EdU for 15 minutes. CAA was performed with H3K27me3 antibody. Following PLA, cells were immunostained for EdU. Lower panel shows PLA signals only. FIG. 4B shows western blot analysis of H3K27me3 in THP-1 cells (left) and AML primary cell extracts (right).

FIG. 5 illustrates the use of EZH2 inhibitor GSK343 increases sensitivity to DNA damage. Cells from AML patient #22324 were treated with the GSK343 EZH2 inhibitor for 12 hours, switching post-replicative chromatin from “closed” to “open” conformation (see illustration below the micrographs). Cells were labeled with EdU for 10 minutes and treated with 10 μM Bleomycin (Bleo) for 1 hr. CAA (PLA, dark) was performed between EdU and ATRIP protein.

FIGS. 6A-6D illustrate EZH2 inhibition enhancing cytotoxic drug-induced DNA damage of THP-1 cells. DNA damage evaluation by analysis of γ-H2A.X foci. Cells were treated with Bleomycin (5 μM), Doxorubicin (0.1 μM) or Etoposide (0.1 μM) alone or in combination with the EZH2 inhibitor GSK-126 (5 μM) for 24 hours. FIG. 6A shows immunofluorescence microscopy of γ-H2A.X foci (representative cells). γ-H2A.X light gray, DAPI, dark gray. Lower panels show γ-H2A.X signals only. FIG. 6B displays quantification of the number of γ-H2A.X foci+/−SEM. t-test was; **p<0.01; ***p<0.001. In FIG. 6C, DNA damage was assessed by γ-H2A.X western blot in drug-treated THP-1 cell extracts. FIG. 6D shows a comet assay measuring DNA damage in drug-treated THP-1 cells. >200 cells analyzed in each condition.

FIG. 7 illustrates the kinetics of RAD51 foci formation in Doxorubicin-treated THP-1 cells. THP-1 cells were left untreated or treated for 12 hrs with Doxorubicin (0.1 μM), GSK126 (5 μM), or Doxorubicin/GSK126 (0.1 and 5 μM, respectively). At the end of the treatment, cells were washed and GSK126 was re-added (5 μM) to the GSK126- or the GSK126/Doxorubicin-treated cultures. RAD51 foci were detected by immunofluorescence in the pre-washed cultures (12 hrs), and at 24, 36, and 72 hrs post Doxorubicin washing.

FIGS. 8A-8D illustrate that inhibition of EZH2 enhances DNA damage as assessed by γ-H2A.X foci and comet assay in primary CD34+ AML cells. Cells were left untreated (CTRL) or treated with GSK126 alone (5 μM; 24 hrs), Doxorubicin alone (0.3 or 0.1 μM for AML #013 and AML #010 respectively; 24 h), or both drugs at the same concentrations. FIG. 8A shows AML #013 cells. Immunofluorescence microscopy of γ-H2A.X foci (representative cells). γ-H2A.X light gray, DAPI, dark gray. Lower panels show γ-H2A.X signals only. FIG. 8B shows AML #010 cells. Immunofluorescence microscopy of γ-H2A.X foci (representative cells). γ-H2A.X light gray, DAPI, dark gray. Lower panels show γ-H2A.X signals only. FIG. 8C displays quantification of the number of γ-H2A.X foci+/−SEM. t-test was; *p<0.05; **p<0.01. FIG. 8D shows a Comet Assay measuring DNA damage in drug-treated primary CD34+ AML cells. >100 cells were analyzed in each condition.

FIGS. 9A-9B illustrate apoptosis of drug-treated cells. THP-1 (FIG. 9A) and primary CD34+ AML samples (FIG. 9B) were left untreated (CNTR), treated with Doxorubicin alone (0.1 μM), Etoposide alone (0.5 μM) or in combination with the EZH2 inhibitor GSK-126 (0.5 μM) for 48 hours. Apoptosis was analyzed by Caspase-3/7 activity by flow cytometry.

FIGS. 10A-10C illustrate that treatment with the CDK4/CDK6 inhibitor palbociclib suppresses Doxorubicin-induced DNA damage. FIG. 10A displays DNA content analysis of untreated and palbociclib-treated (500 nM; 24 h) THP-1 cells. FIG. 10B is a series of images showing immunofluorescence microscopy of γ-H2A.X foci in untreated or drug-treated (Doxorubicin 0.1 μM, palbociclib 500 nM) THP-1 cells. γ-H2A.X light gray, DAPI dark gray. In FIG. 10C, apoptosis was analyzed by Caspase 3/7 activity by flow cytometry.

FIGS. 11A-11B illustrate that treatment with GSK126 alone had no effect on proliferation or differentiation of AML cells. FIG. 11A displays DNA content analysis of untreated or GSK126-treated (48 hrs) THP-1 and human CD34+ primary AML cells. EZH2i did not change the % of cycling cells. FIG. 11B shows a flow cytometry analysis of myeloid marker expression in GSK126-treated (5 days) AML #013 cells. Representative of three GSK126-treated AML samples.

FIGS. 12A-12C illustrate the THP-1 leukemia burden and survival in mice treated with chemotherapy and/or EZH2 inhibitor GSK126. In FIG. 12A, a diagram illustrating the schedule of drug treatments in a THP-1-Luciferase xenograft model is displayed. FIG. 12B: NOD-Rag 1-/-IL2Rγ-null (NRG) mice were injected with 1×10⁶ cells and treated 5 days later with GSK126 (100 mg/kg/daily for 7 days), Doxorubicin/Ara-C (5+3; 1.5 mg/kg Doxorubicin/3 days/50 mg/kg Ara-C/5 consecutive days), or the combination. Serial bioluminescence images of mice acquired 1 and 2 weeks after the end of the treatment. FIG. 12C shows Kaplan-Meier survival curves of untreated (n=7), GSK126-treated (n=9), Doxorubicin/Ara-C-treated (n=10), and Doxorubicin/Ara-C/GSK126-treated (n=10) mice.

FIGS. 13A-13B illustrate accumulation of H3K27me3 on nascent DNA of a human primary ASXL1 mutated AML sample. In FIG. 13A, DNA was labeled with EdU for 15 minutes. Following conjugation with biotin, CAA was performed between nascent DNA (biotin) and H3K27me3. PLA (bright gray), Edu (biotin; light gray), DAPI; dark gray). Lower panels show PLA signals only. (Right) treatment with GSK126 does not enhance DNA damage of a primary ASXL1-mutated AML sample. DNA Damage evaluation by analysis of γ-H2A.X foci. Cells were treated with Doxorubicin alone or in combination with the EZH2 inhibitor GSK126 (5 nM) for 24 hours. Immunofluorescence microscopy of γ-H2A.X foci (representative cells). γ-H2A.X light gray, DAPI, dark gray. Lower panels show γ-H2A.X signals only. FIG. 13B shows quantification of the number of γ-H2A.X foci+/−SEM.

FIGS. 14A-14C illustrate the effects of GSK126 treatment. In FIG. 14A, treatment of THP-1 cells with GSK126 for 24 hours reduces global levels of H3K27me3. Box plot of ChIP-seq read density is shown. FIG. 14B is an example showing the COL93A locus. A ChIP-seq of γ-H2AX in untreated and drug-treated THP-1 cells is shown in FIG. 14C.

FIG. 15 illustrates that synchronization in the S-phase and treatment of cells with the EZH2 inhibitor GSK126 further enhances Doxorubicin-induced DNA Damage and apoptosis. THP-1 cells were treated with Palbociclib (500 nM) for 24 hours to induce a G1-arrest. Palbociclib was washed out and THP-1 cells re-entering S-phase were treated with GSK126 (5 μM) for 12 hours, before adding the Doxorubicin (0.1 μM); (Left) Quantification of the number of g-H2A.X foci+/−SEM. Anova ***p<0.001; (Right) Apoptosis analyzed by Caspase 3/7 activity by flow cytometry. Mean+/−SEM. Anova; *p<0.1 **p<0.01

FIG. 16 illustrates the leukemia burden of NRG mice injected with THP-1 cells, pre-treated with Palbociclib and then treated with chemotherapy and/or GSK126. (Top) NRG mice were injected with THP-1-Luc+ cells (1×10⁶ cells/mouse). 5 days post-injection, mice were fed with chow containing 150 mg/kg of Palbociclib for 3 days. Then, mice were left untreated (Palb group), treated with GSK126 alone for 7 days (Palb/EZH2i group) or pre-treated (2 days) with GSK126 and then co-treated with GSK126 and chemotherapy for 5 additional days (Doxo/Ara-C/EZH2i group); the Palb/Doxo/Ara-C only group was treated for 5 days, starting 10 days after THP-1 cells injection (Bottom) Serial bioluminescence images of mice were acquired 2 and 3 weeks after the end of the treatment.

FIG. 17 illustrates the leukemia burden of Doxo/Ara-C- vs GSK126/Doxo/Ara-C-treated NRG-SGM3 mice injected with AML cells. Mice were injected with AML sample #040 (1.5×10⁶ cells/mouse). When peripheral blood human CD45-positive cells were >10%, animals were divided in 4 groups and treated with vehicle, GSK126, Doxo/Ara-C, or the Doxo/Ara-C/GSK126 combination. Leukemia burden was then assessed by measuring the % of peripheral blood leukemic cells by anti-human CD45-FITC flow cytometry at one-week intervals starting 10 days after therapy cessation. Axes indicate the fold changes of the percentage of leukemic cells in pre-treated vs treated mice in each mouse cohort. Error bars indicate standard deviation.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “agonist” refers to a substance that acts like another substance and therefore stimulates an action. For example, an agonist can be a molecule capable of binding a specific protein and initiating the same reaction or activity typically produced by the binding endogenous substance. An agonist can be any molecule that increases transcription, increases translation, or increases the activity of the cognate molecule.

The term “cleavage” refers to the breakage of covalent bonds, such as in the backbone of a nucleic acid molecule. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “transgene” refers to the genetic material that has been or is about to be artificially inserted into the genome of an animal, particularly a mammal and more particularly a mammalian cell of a living animal.

To “treat” a disease as the term is used herein, means to reduce the frequency or severity of at least one sign or symptom of a disease or disorder experienced by a subject.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

As used herein, “therapeutic index” refers to the ratio of the toxic dose, or dose of a drug that causes adverse effects incompatible with effective treatment of the disease or condition, to the effective dose, or dose of a drug that leads to the desired therapeutic effect in treatment of the disease or condition.

As used herein, the term “chemotherapy” refers to a clinical treatment strategy used to treat cancer using chemicals and compounds that are preferentially toxic to the cancer cells. Chemotherapy may be given to cure a particular cancer, or it may be used to slow the progression of disease or reduce symptoms of disease. The preferential toxicity of chemotherapeutic agents for cancer cells is typically conferred through the targeting of processes or proteins relatively unique to the cancer cells. Mechanisms of such targeting can range from generic targeting of rapidly-dividing cells, for instance through the use of poisons and inhibitors of cell division, to protein-specific targeting to inhibit gene expression, cell growth, cell function, or induce cell death.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

Recent studies have identified that normal CD34+ hematopoietic progenitor cells (HPCs) are characterized by a significant period during which recently-replicated chromatin is globally devoid of the repressive histone mark H3K27me3. This type of de-condensed chromatin is required for recruitment of lineage-determining transcription factors (TFs) such as C/EBPα, PU.1 and GATA-1. HPC reception of cytokine based differentiation-promoting signals induce these transcription factors to bind their target sequences on the nascent DNA and initiate erythroid or myeloid differentiation programs. Increasing H3K27me3 on nascent DNA by pharmacological inhibition of the UTX/JMJD3 H3K27me3 demethylases prevents recruitment of these TFs to the DNA, and suppresses G-CSF- or EPO-induced differentiation. By contrast, blasts from several adult acute myeloid leukemia (AML) patients exhibit a markedly “condensed” post-replicative chromatin characterized by the rapid association of H3K27me3 with nascent DNA. This altered structure of post-replicative chromatin may decrease the efficiency of the DNA damage response in AML blasts.

Understanding the role of nascent chromatin in DNA damage is important because current anti-cancer therapies include chemotherapeutic agents which target cells during DNA replication, and induce DNA damage-dependent apoptosis. Moreover, DNA damage causes primary and secondary cancers and contributes to many of the side effects of cancer therapy. It is likely that a more condensed structure of nucleosomes protects DNA from damage. For example, heterochromatin is more resistant to formation of double-strand breaks, while HDAC inhibitors, which cause chromatin relaxation, increase sensitivity of cancer cell lines to ionizing radiation (IR). More recently, chemical profiling of the genome with DNA-damaging cytotoxic agents has shown that daunomycin, the closely related doxorubicin, and etoposide do not induce double-strand breaks (DSBs) randomly but act preferentially at transcriptionally active sites, i.e. regions of the genome with de-condensed chromatin, as indicated by co-localization of γ-H2A.X with histone marks H3K36me3 and H3K79me2 and with Pol2B. Hence, the relationship between chromatin structure and DNA damage can be used to optimize anti-cancer therapies. In the current invention, epigenetic inhibitors are be used as modifiers of global post-replicative chromatin structure to enhance the cytotoxic effects of DNA damaging agents.

### Histone Modification and Chromatin Structure

Long eukaryotic genomic DNA molecules are organized and packaged into dense structures collectively called chromatin by interaction with protein complexes called histones. The strands of DNA are wound around histones to form complexes called a nucleosomes in a manner similar to thread wound around a spool. Each histone complex, also called a core particle, is an octamer of two copies each of four histone proteins H2A, H2B, H3, and H4 around which 146 base pairs of DNA are wrapped in 1.67 left-handed super-helical turns. Core particles are separated by regions of linker DNA that vary in length between ˜10-90 base pairs. The packaging of DNA into tight chromatin structures can affect expression of the encoded genes by physically preventing access of transcription factor and enhancer proteins to the DNA molecule. Regions of DNA undergoing active transcription are observed to have a loose, “open” structure, while less active regions feature more tightly-packed nucleosomes, often compacted into a larger chromatin structure called the 30 nm fiber. Histone proteins can themselves undergo chemical modification in the form of methylation, phosphorylation, acetylation, ubiquitination, and sumoylation, among others. These modifications can, in turn, affect the ability of the histones to assume “open” and “closed” structures, thereby affecting transcription. In this way, chemical modification of histones acts as a type of epigenetic gene regulation.

A non-limiting example of such histone modification is the addition of three methyl groups to Lysine 27 of histone H3 to form the H3K27 histone mark. This modification is associated with significant suppression of gene transcription activity, and as such is highly associated with inactive genes, especially developmental genes and X-chromosome inactivation. H3K27me3 is unique among histone methylation marks in that only one known methyltransferase enzyme is responsible for catalyzing this modification. EZH2, or enhancer of zeste homolog 2, is a histone-lysine-N-methyltransferase that is part of the PRC2 complex. In some embodiments of the current invention, a histone methyltransferase inhibitor is used in combination with a cell cycle inhibitor and a chemotherapy in order to treat cancer in a subject in need thereof. In some embodiments, the histone methyltransferase inhibitor is an inhibitor of the methylation of any one or both of H3K27 and H3K9. In one embodiment, the histone methyltransferase enzyme which is the target of inhibition is Enhancer of Zeste Homolog 2 (EZH2).

That a single methyltransferase enzyme is responsible for regulation of such a key repressor of gene expression makes EZH2 an attractive target for small-molecule inhibitor development. Examples of EZH2 inhibitors include but are not limited to tazemetostat, GSK126, 3-deazaneplanocin A (DZNep), EPZ005687, CPI-1205, and GSK343.

DZNep inhibits the hydrolysis of S-adenosyl-L-homocysteine (SAH), which is a product-based inhibitor of all protein methyltransferases, leading to increased cellular concentrations of SAH which in turn inhibits EZH2. However, DZNep is not specific to EZH2 and also inhibits other DNA methyltransferases.

EPZ005687, an S-adenosylmethionine (SAM) competitive inhibitor that is more selective than DZNep; it has a 50-fold increase in selectivity for EZH2 compared to EZH1. The drug blocks EZH2 activity by binding to the SET domain active site of the enzyme. EPZ005687 can also inhibit the Y641 and A677 mutants of EZH2, which may be applicable for treating non-Hodgkin's lymphoma.[41] In 2013, Epizyme began Phase I clinical trials with another EZH2 inhibitor, tazemetostat (EPZ-6438), for patients with B-cell lymphoma.[45] In 2020, tazemetostat, with the tradename Tazverik, was FDA approved for the treatment of metastatic or locally advanced epithelioid sarcoma and was approved for the treatment of patients with relapsed follicular lymphoma later that year[46].

GSK126 is an S-adenosylmethionine (SAM) competitor, and is highly selective for the EZH2 enzyme with a Ki value of ˜0.5 nM. In one embodiment of the current invention, the EZH2 inhibitor is GSK126.

### Cell Cycle Inhibitors

The regulation of the cell cycle is governed and controlled by specific proteins, which are regulated mainly through phosphorylation/dephosphorylation processes in a precisely timed manners. The key proteins that coordinate the initiation, progression, and completion of cell-cycle programs are cyclin dependent kinases (CDKs). Cyclin-dependent kinases belong to the serine-threonine protein kinase family, and are heterodimeric complexes composed of a catalytic kinase subunit and a regulatory cyclin subunit. CDK activity is controlled by association with their corresponding regulatory subunits (cyclins) and other CDK inhibitor proteins (Cip & Kip proteins, INK4, among others), by their phosphorylation state, and by ubiquitin-mediated proteolytic degradation (see D. G. Johnson, C. L. Walker, Annu. Rev. Pharmacol. Toxicol 39 (1999) 295-312; D. O. Morgan, Annu. Rev. Cell Dev. Biol. 13 (1997) 261-291; C. J. Sherr, Science 274 (1996) 1672-1677; T. Shimamura et al., Bioorg. Med. Chem. Lett. 16 (2006) 3751-3754).

There are four CDKs that are significantly involved in controlling cellular proliferation: CDK1, which predominantly regulates the transition from the second growth phase (G2) to the mitosis phase of active cell division (M phase), and CDK2, CDK4, and CDK6, which regulate the transition from the first growth phase (G1) to the DNA synthesis phase (S phase). In early to mid G1 phase, when the cell is responsive to differentiation-inducing mitogenic stimuli, activation of CDK4-cyclin D and CDK6-cyclin D induces phosphorylation of the retinoblastoma protein (pRb). Phosphorylation of pRb releases the transcription factor E2F, which enters the nucleus and activates transcription of other cyclins which promote further progression of the cell cycle (see J. A. Diehl, Cancer Biol. Ther. 1 (2002) 226-231; C. J. Sherr, Cell 73 (1993) 1059-1065). CDK4 and CDK6 are closely related proteins with basically indistinguishable biochemical properties (M. Malumbres, M. Barbacid, Trends Biochem. Sci. 30 (2005) 630-641).

In certain embodiments of the current invention, the cell cycle inhibitor is an inhibitor of both CDK4 and CDK6. A number of CDK 4/6 inhibitors have been identified, including specific pyrido[2,3-d]pyrimidines, 2-anilinopyrimidines, diaryl ureas, benzoyl-2,4-diaminothiazoles, indolo[6,7-a]pyrrolo[3,4-c]carbazoles, and oxindoles. For example, 6-acetyl-8-cyclopentyl-5-methyl-2-(5-piperazin-1-yl-pyridin-2-ylammino)-8H-pyrido-[2,3-d]-pyrimidin-7-one (PD0332991), also called palbociclib, is currently FDA approved for estrogen receptor positive (ER+) advanced breast cancer and hormone receptor positive (HR+), HER2 negative advanced or metastatic breast cancer. Examples of other CDK4/6 inhibitors include, but are not limited to abemaciclib (LY2835219), ribociclib (LEE011), and iodine-containing pyrido[2,3-d]pyrimidine-7-one (CKIA).

### DNA-Damaging Chemotherapy Treatment of Cancer

DNA damaging agents or factors are chemotherapeutic compounds that induce harmful chemical modifications or damage to DNA molecules when applied to a cell, and have a long history of use in treating cancers. As a cancer treatment, these compounds lead to the induction of cell death in rapidly-dividing cells.

In certain embodiments of the current invention, the chemotherapy is a topoisomerase II inhibitor. Topoisomerase inhibitors are drugs that affect the activity of two enzymes: topoisomerase I and topoisomerase II. When the double-stranded helix structure of DNA is unwound, such as during DNA replication or transcription, the adjacent unopened coils of DNA wind tighter (supercoils). The physical resistance caused by this unwinding would inhibit the further interaction of replication or transcription complexes with the DNA, however topoisomerase enzymes act to relive the tension on the DNA molecules by producing single- or double-strand breaks in the DNA strands, thereby releasing the supercoiling and reducing the tension. Inhibition of topoisomerase I or II interferes with both of these processes and inhibits DNA replication, and ultimately, cell proliferation. Two topoisomerase I inhibitors, irinotecan and topotecan, are semi-synthetically derived from camptothecin, which is obtained from the Chinese ornamental tree Camptotheca acuminate. Drugs that target topoisomerase II can be divided into two groups: poisons and inhibitors. The topoisomerase II poisons inhibit specific steps in the process by which topoisomerase II releases DNA tension. For example, etoposide and teniposide prevent the re-ligation of DNA after the topoisomerase II-induced cleavage, resulting in strand breaks, leading to programmed cell death (apoptosis). These agents include etoposide, doxorubicin, mitoxantrone and teniposide. The second group, catalytic inhibitors, are drugs that block the activity of topoisomerase II by targeting the N-terminal ATP domain and prevent turnover, and therefore prevent DNA synthesis and translation because the DNA cannot unwind properly. This group includes novobiocin, merbarone, ICRF-193, genistein, and aclarubicin.

Many of the aforementioned topoisomerase II-targeting drugs also have other significant mechanisms of action, particularly those of the anthracycline class, which include daunorubicin, doxorubicin, epirubicin, and indarubicin. In addition to activity as topoisomerase II poisons, these compounds can also intercalate, or insert, into the DNA molecule itself thus blocking DNA and RNA synthesis machinery. Additionally, they can lead to the production of excess reactive oxygen species (ROS) within the target cell through redox reactions involving the quinone moiety and cytochrome p450 reductase, NADH dehydrogenase, and xanthine oxidase enzymes. The excessive ROS leads to DNA damage, lipid peroxidation, and ultimately senescence or apoptosis. Anthracyclines can also result in DNA adducts that also disrupt DNA synthesis and transcription.

In certain embodiments, the chemotherapy may induce chemical cross-linking of the DNA molecules. One such class of DNA cross-linking drugs are platinum-based drugs, or platins. These molecules are coordination complexes containing platinum atoms that cause crosslinking as monoadducts, insterstrand crosslinks, intrastrand crosslinks, or DNA/protein crosslinks, mostly at the N-7 position of guanine. The resulting crosslinks inhibit DNA repair and DNA synthesis, leading to cell death. Non-limiting examples of platinum-based drugs include cisplatin, oxaliplatin, carboplatin, lipoplatin, and nedaplatin,

In certain embodiments of the current invention, the cytotoic chemotherapy is an antimetabolite. Drugs of this class interfere with one or more enzymes that mediate the synthesis of DNA by acting as substitutes to the actual metabolites that would be normally used. Antimetabolites actively compete with the normal metabolite for access to the target enzyme, resulting in toxic effects to the cell. Types of antimetabolites include base analogs that can substitute for normal nucleotide bases in DNA and RNA. Analogs of purines include but are not limited to azathioprine, thiopurine, and fludarabine including analogs of those moleucles. Analogs of pyrimidines include 5-fluorouracil, gemcitabine, and cytarabine, as well as their analogs.

In certain embodiments of the current invention, the chemotherapy is bleomycin. Bleomycin is a non-ribosomal peptide antibiotic that also causes DNA strand breaks by a poorly understood mechanism. Bleomycin was first discovered in 1962 as a product of the bacteria Streptomyces verticillus that had anti-cancer activity.

### Methods of Treatment

In certain aspects, the invention includes a method of treating cancer in a subject comprising administering to the subject an effective amount of a histone methyltransferase inhibitor, and an effective amount of a chemotherapy. In certain embodiments, the method further comprises administering to the subject an effective amount of a cell cycle inhibitor.

In some embodiments, the cell cycle inhibitor is administered first, followed by the histone methyltransferase inhibitor, followed by the chemotherapy. In some embodiments, the cell cycle inhibitor is administered first, followed by concurrent administration of the histone methyltransferase inhibitor and chemotherapy. In some embodiments, the cell cycle inhibitor and histone methyltransferase inhibitor are delivered concurrently followed by the chemotherapy. In further embodiments, the cell cycle inhibitor, histone methyltransferase inhibitor, and chemotherapy are delivered concurrently.

In another aspect, the invention includes a method of treating cancer in a subject, comprising: a) administering to a sample from the subject, a plurality of combinations of histone methyltransferase inhibitors and chemotherapies, b) determining the optimal combination of histone methyltransferase inhibitors and chemotherapies, and c) treating the subject with the optimal combination of histone methyltransferase inhibitors and chemotherapies.

Determining the optimal combination of histone methyltransferase inhibitors and chemotherapies can comprise measuring from a tumor sample from a subject one or more parameters of the loss of repressive histone marks, DNA damage, and/or cytotoxicity using assays known to one of ordinary skill in the art, for example cytotoxicity assays, cytokine assays, apoptosis assay (e.g. Annexin V), DNA damage assays, and the like.

In some embodiments, the subject is a human. In some embodiments, the cancer is a hematologic malignancy. The term hematologic malignancy broadly refers to pathologic conditions which involve any of the many cell types that are of hematopoietic origin including lymphocytes and myeloid lineage cells. The diversity in the lineages and differentiation stages of hematopoietic cells results in a large number of distinct and heterogeneous tumors generally referred to as hematologic malignancies. Thus, hematologic malignancies or hematologic neoplasia affect cells and tissues of the blood, including blood, bone marrow and lymph nodes. Hematologic malignancies include both leukemias and lymphomas. The term leukemia has generally been used to define hematologic malignancies of the blood or bone marrow characterized by abnormal proliferation of leukocytes. The principal subtypes of leukemia are identified on the basis of malignancy involving lymphoid (e.g. T or B lymphocytic lineage) or myeloid (e.g. granulocytic, erythroid or megakaryocytic lineage) cells, and whether the disease is acute or chronic in onset. The term lymphoma covers a heterogeneous group of neoplasms of lymphoid tissue. Lymphomas are broadly categorized under Hodgkin lymphoma, and T-cell (T-NHL) and B-cell (B-NHL) non-Hodgkin lymphomas. A World Health Organization (WHO) classification has recently been published and diagnostic guidelines have been established based on this classification. In certain embodiments of the current invention, examples of hematologic malignancies include but are not limited to acute myelogenous leukemia and acute non-lymphocytic leukemia, such as acute megakaryoblastic leukemia and acute erythroblastic leukemia. In one embodiment, the hematologic malignancy is acute myelogenous leukemia. In one embodiment, the hematologic malignancy is chronic myelogenous leukemia-blast crisis.

### Pharmaceutical Compositions

The dosage of a cell cycle inhibitor to be administered to a patient may be from about 0.1 to about 100 mg/m². In certain embodiments, the dosage may be from about 0.1 to about 70 mg/m². In certain embodiments, the dosage may be from about 0.1 to about 60 mg/m². In some embodiments, the dosage may be from about 0.1 to about 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m². In some embodiments, the dosage may be about 0.1, 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m².

The dosage of a histone methyltransferase inhibitor to be administered to a patient may be from about 0.1 to about 100 mg/m². In certain embodiments, the dosage may be from about 0.1 to about 70 mg/m². In certain embodiments, the dosage may be from about 0.1 to about 60 mg/m². In some embodiments, the dosage may be from about 0.1 to about 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m². In some embodiments, the dosage may be about 0.1, 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m².

The dosage of a chemotherapy to be administered to a patient may be from about 0.1 to about 100 mg/m². In certain embodiments, the dosage may be from about 0.1 to about 70 mg/m². In certain embodiments, the dosage may be from about 0.1 to about 60 mg/m². In some embodiments, the dosage may be from about 0.1 to about 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m². In some embodiments, the dosage may be about 0.1, 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m².

Human dosage amounts can initially be determined by extrapolating from the amount of compound used in mice, as a skilled artisan recognizes it is routine in the art to modify the dosage for humans compared to animal models. In certain embodiments it is envisioned that the dosage may include an effective amount from between about 0.001 mg compound/Kg body weight to about 100 mg compound/Kg body weight; or from about 0.05 mg/Kg body weight to about 75 mg/Kg body weight or from about 0.1 mg/Kg body weight to about 50 mg/Kg body weight; or from about 0.5 mg/Kg body weight to about 40 mg/Kg body weight; or from about 0.1 mg/Kg body weight to about 30 mg/Kg body weight; or from about 1 mg/Kg body weight to about 20 mg/Kg body weight. In other embodiments, the effective amount may be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, or about 100 mg/Kg body weight. In other embodiments, it is envisaged that effective amounts may be in the range of about 2 mg compound to about 100 mg compound. In other embodiments, the effective amount may be about 1, 2, 3, 4, 5, 6, 7, 8, 9, 10, 11, 12, 13, 14, 15, 16, 17, 18, 19, or 20 mg per single dose. In another embodiment, the effective amount comprises less than about 2, 3, 4, 5, 6, 7, 8, 9, 10, 15, 20, 25, 30, 35, 40, 45, 50, 55, 60, 65, 70, 75, 80, 85, 90, 95 mg daily. In an exemplary embodiment, the effective amount comprises less than about 50 mg daily. Of course, the single dosage amount or daily dosage amount may be adjusted upward or downward, as is routinely done in such treatment protocols, depending on the results of the initial clinical trials and the needs of a particular patient.

Administration of the methods of treatment of the present invention to a patient, preferably a mammal, more preferably a human, may be carried out using known procedures, at dosages and for periods of time effective to treat a disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder in the patient. Dosage regimens may be adjusted to provide the optimum therapeutic response. For example, several divided doses may be administered daily or the dose may be proportionally reduced as indicated by the exigencies of the therapeutic situation.

The precise determination of what would be considered an effective dose is based on factors individual to each subject, including their size, age, sex, weight, and condition of the particular subject. Dosages can be readily ascertained by those skilled in the art from this disclosure and the knowledge in the art.

Optionally, the methods of the invention provide for the administration to a suitable animal model to identify the dosage of the composition(s), concentration of components therein and timing of administering the composition(s), which elicit the desired biological response, e.g., prevention or reduction of a fibropolycystic disease. Such determinations do not require undue experimentation, but are routine and can be ascertained without undue experimentation.

In certain embodiments, the methods of treatment of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

The biologically active agents can be conveniently provided to a subject as sterile liquid preparations, e.g., isotonic aqueous solutions, suspensions, emulsions, dispersions, or viscous compositions, which may be buffered to a selected pH. AAgents of the invention may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by suspending a FXR inhibitor in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like.

Routes of administration of any of the methods of treatment of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable formulations and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

For parenteral administration, the methods of treatment of the invention maybe formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Additional Administration Forms

In various embodiments, the methods of treatment of the invention may be delivered transdermally. In various embodiments, the transdermal delivery formulation may contain one or more penetration enhancers.

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer than the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In certain embodiments of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Methods

Cell lines and culture conditions: THP-1 is a leukemia line derived from a patient with monocytic leukemia. This cell line is characterized by expression of the oncogenic chimeric protein MLL-AF9 resulting from the t(11;9) translocation. THP1 cells grow in suspension in a medium consisting of RPMI 1640+10% FBS+2 mM L-Glutamine. The average doubling time is 19 to 50 hours. 1 mM sodium pyruvate, penicillin (100 units/ml) and streptomycin (100 μg/ml) are also commonly added to inhibit bacterial contamination. Cultures should be maintained at cell densities in the range 2-9×10⁵ cells/ml at 37° C., 5% CO₂. The Luciferase-positive THP1 cell line was derived from the parental THP1 cell line by lentiviral transduction with a vector expressing the luciferase protein. Expression of this protein is needed to detect leukemia in mice by bio-imaging. Culture conditions of the LUC+-THP1 cell line are identical to the parental THP1 line.

Inhibitors and chemotherapies: The EZH1/EZH2 inhibitor GSK126 and GSK343 are used at a concentration of 1-5 μM. The H3K27 methyltransferase inhibitor UNC0638 is used at a concentration of 1-5 μM. Palbociclib is used at 250-500 nanomolar. Doxorubicin is used ex vivo at 0.1-0.5 μM. Etoposide is used at 0.1-0.5 μM. Bleomycin is used at 5 μM. For in vivo studies in NSG mice, GSK126 is used at 100 mg/Kg/IP/7 days; Doxorubicin is used at 60 mg/Kg/IV, 3 days; Cytosine Arabinoside (Ara-C; generic name: cytarabine) is used at 60 mg/Kg/IP, 5 days. Palbociclib is used at 150 mg/Kg/IP, 3 days.

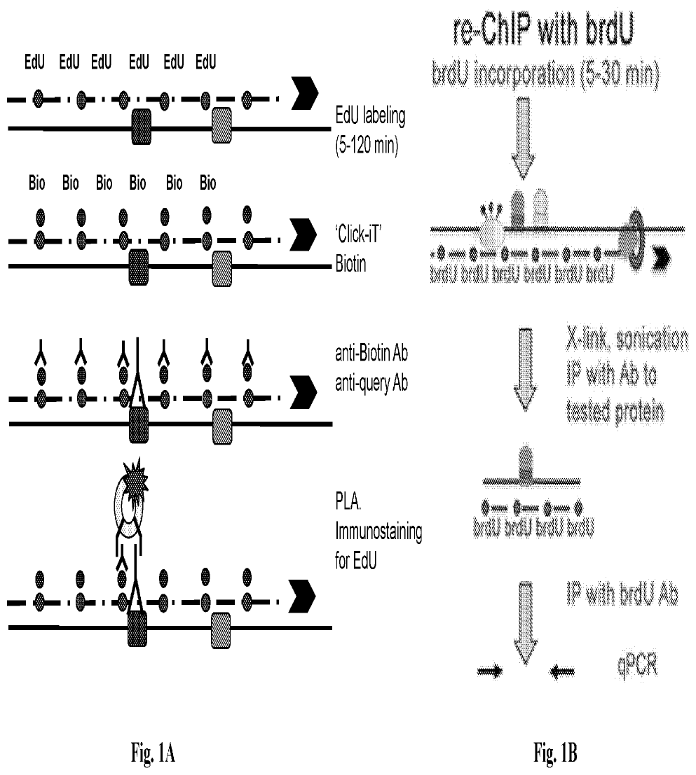

Chromatin assembly assay: Cells were grown on chamber slides, pulse-labeled with 5 μM EdU and fixed at room temperature with 4% formaldehyde in PBS for 15 min, washed with PBS, and permeabilized with 0.3% Triton for 15 min. Cells were subjected to Click-iT reaction with biotin-azide for 30 min. The PLA reactions (Olink) between the anti-biotin antibody and antibodies to other proteins were performed as described by Olink. Following PLA, cells were immunostained with anti-biotin Alexa Fluor 488 antibody to control the specificity of CAA. The results of CAA experiments shown in FIG. 1A were quantified by counting the number of PLA signals per EdU-labeled nuclei in 50 cells of each of the three independent experiments. The nature of the results in the rest of the figures alleviated the need to quantify the results of these assays.

Re-ChIP assay: 2-4×10⁷ cells were grown on 100 mm dishes, and half of the cells were induced for 2 hr 30 min with the mDA cocktail. Undifferentiated and induced cells were labeled with 50 μM BrdU for 12 min or 12 min followed by chase to 60 min, washed with PBS and fixed with 1% formaldehyde for 10 min at room temperature. After fixation glycine was added to a final concentration of 0.125 M to quench formaldehyde. Cells were washed with PBS, scraped from plates, collected by centrifugation for 5 min at 1000 rpm at 4° C., re-suspended in 1 ml of RIPA buffer (1% Triton X-100, 0.1% sodium deoxycholate, 0.05% SDS, 0.15 M NaCl, 10 mM Tris-HCl, pH 8.0, 1 mM EDTA, pH 8.0) with protease inhibitor. Suspension was sonicated to shear DNA to an average length of about 500-1000 bp and centrifuged for 5 min at 14,000 rpm. The supernatant was pre-cleared with protein-G agarose/Salmon Sperm DNA for 1 h at 4° C. 5% of material was used as input, and the remaining material was divided for incubation overnight at 4° C. with 30 μg of rabbit polyclonal anti-trimethyl H3K27 or with 30 μg of rabbit IgG as a control. The protein-G agarose/Salmon Sperm DNA was added for 1 h, beads were collected by centrifugation and sequentially washed for 3 min in 1 ml of low salt wash buffer (0.1% SDS, 1% Triton-X100, 2 mM EDTA, 20 mM Tris-HCl, pH 8.1, 150 mM NaCl), high salt wash buffer (0.1% SDS, 1% TritonX-100, 2 mM EDTA, 20 mM Tris-HCl, pH 8.1, 500 mM NaCl), LiCl wash buffer (0.25 M LiCl, 1% NP40, 1% deoxycholate, 1 mM EDTA, 10 mM Tris-HCl pH 8.1) and then twice with TE (10 mM Tris-HCl pH, 8.0, 1 mM EDTA). Beads were incubated two times for 15 min in 250 μl of the elution buffer (1% SDS, 0.1 M NaHCO3) at room temperature, supernatants were combined and incubated at 65° C. for 16 h in the presence of 0.2 M NaCl to reverse cross-link, and treated with proteinase K for 2 h at 45° C. DNA was purified by phenol/chloroform extraction and precipitated with ethanol. DNA was re-suspended in 500 μl of the TE buffer. 5% of material was removed and used as second input in the analysis by real time PCR. 20 μg of salmon sperm DNA was added to the remaining material. Samples were boiled for 5 min and then kept on ice for 2 min. 50 μl of the 10× adjusting buffer (110 mM sodium phosphate, pH 7.0, 1.52 M NaCl, 0.55% Triton X100) was added to samples. Samples were incubated at room temperature for 20 min with 15 μg of anti-brdU antibody (BD Biosciences), followed by 20 min incubation with 35 μg of rabbit anti-mouse IgG. Samples were centrifuged at 18,000 g for 20 min and pellet was washed with the adjusting buffer and incubated for 2 h at 45° C. in the lysis buffer (50 mM Tris-HCl, pH 8.0, 10 mM EDTA, 0.5% SDS, 25 mg/ml proteinase K). DNA was purified by phenol/chloroform, precipitated with ethanol and used for analysis. All PCR reactions were performed with an Applied Biosystems StepOne Real-Time PCR system.

Block of DNA replication: To block DNA replication, cells were labeled with EdU for 30 min, induced to specific lineage for 24 hr in the presence of 2 mM thymidine. To restore DNA replication, thymidine was removed by washing, and cells were grown in culture media without thymidine for 4 hr, fixed, and analyzed by CAA with antibodies against biotin (EdU-labeled DNA) and corresponding TFs. The efficiency of the thymidine block of DNA replication was tested by labeling cells with 30 μM BrdU for 20 min in thymidine and after removal of thymidine, fixed with 4% formaldehyde, denatured in 2N hydrochloric acid for 20 min, washed in PBS and incubated with monoclonal anti-BrdU antibody (Exbio) followed by incubation with anti-mouse Alexa Fluor 488.

Gene expression analysis by RT-qPCR: Total RNA was isolated directly from freshly collected hESCs with TRIzol and for mESCs with High Pure RNA Isolation Kit. cDNA was synthesized by using 1 μg total RNA in a 20 μL reaction with Superscript III and oligo (dT) for hESC or random hexamers (Life Tech) for mECS. After reverse transcription was complete, one microliter of RNase H was added to each reaction tube, and the tubes were incubated for 20 min at 37° C. before proceeding to PCR. Real-time PCR was carried out on a 7500 Real-Time PCR System using the 2× Power SYBR green PCR master mix. GAPDH was used as an internal control. All PCR products were checked by running an agarose gel for the first time and by doing dissociation assay every time to exclude the possibility of multiple products. PCR analyses were conducted in triplicate for each sample.

Dilutions of antibodies used: For PLA:rabbit anti SIP1 (1:200), rabbit anti LMX1A (1:200), goat anti FOXA2 (1:200), rabbit anti-RAR beta (1:200), rabbit anti-HOXA5 (1:200), rabbit anti-HOXA3 (1:100), rabbit anti-H3K27me3 (1:2500), mouse anti-biotin for hESCs (1:1000), goat anti-biotin for mESC (1:1000), rabbit anti-UTX (1:400), rabbit ani-JMJD3 (1:400), rabbit anti-EZH2 (1:400), mouse anti-BrdU (1:100).

### Example 1

### Assaying the Fate of Chromatin-Associated Proteins Following DNA Replication

New approaches to study the fate of chromatic-associated proteins following DNA replication were developed. In particular, the Chromatin Assembly Assay (CAA) examines the assembly of chromosomal proteins on nascent DNA at a single-cell level (FIG. 1A). In this assay, DNA is labeled in living cells in a pulse-chase manner with EdU, which is then chemically conjugated with biotin using a ‘Click-iT’ reaction. The proximity of a protein of interest to nascent DNA is then examined by the Proximity Ligation Assay (PLA, Olink, Bioscience) using antibodies to biotin and the protein of interest. PLA is a powerful technique that detects single molecule interactions. Cells can be then examined for specificity of interactions (e.g. specific PLA signals are detected only in EdU-labeled nuclei), and by assessing tissue specificity using relevant protein markers. Reliable pulse-chase EdU labeling in cells can be detected from 3-5 min to several hours. A gene-specific re-ChIP assay with BrdU-labeled DNA, previously used in Drosophila and in human HPCs (FIG. 1B), complements this CAA approach. In this assay, DNA is pulse-labeled with BrdU, chromatin is precipitated with the antibody of interest, and DNA is then denatured and precipitated with antibody against BrdU, followed by qPCR. This method allows the examination of the accumulation of proteins at specific binding sites over time after DNA replication. With both assays, a detailed picture of the behavior of proteins at their DNA binding sites at the single-cell level during and after DNA replication can be obtained.

### Example 2

### Dynamics of Accumulation of H3K27me3 on Nascent DNA of Multipotent Hematopoietic Progenitors (HPCs)

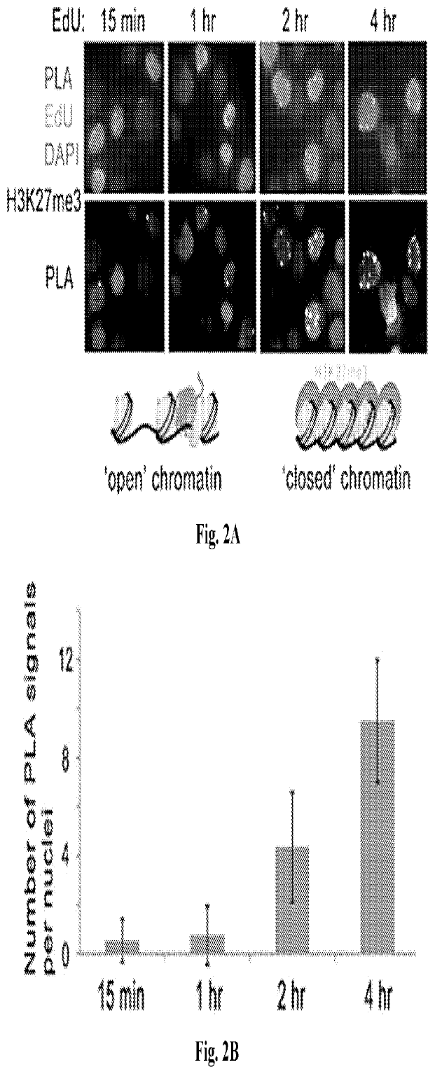

Using the CAA, it was found that accumulation of post-replicative H3K27me3 on nascent DNA of normal CD34+CD38+ HPCs is significantly delayed (2-4 hrs after DNA replication) (FIGS. 2A and 2B). This de-condensed structure of nascent chromatin is essential for the association of lineage-determining transcription factors (TFs) with repressed genes.

### Example 3

### Accumulation of H3K27me3 in AML Cells

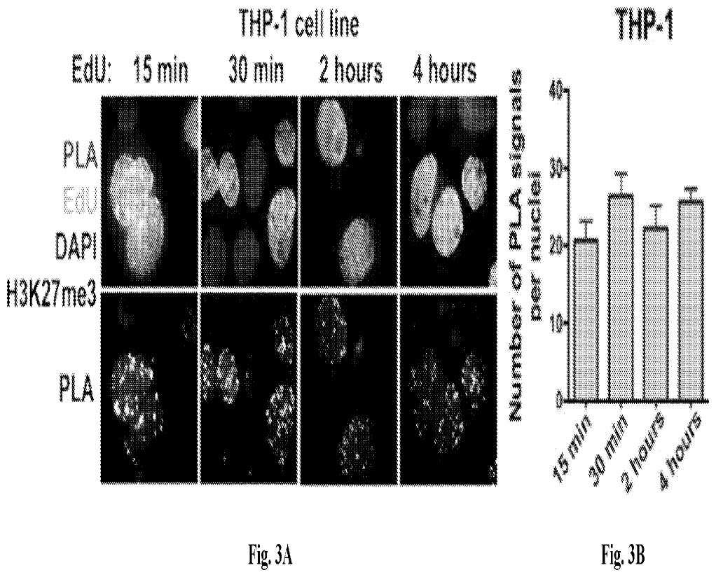

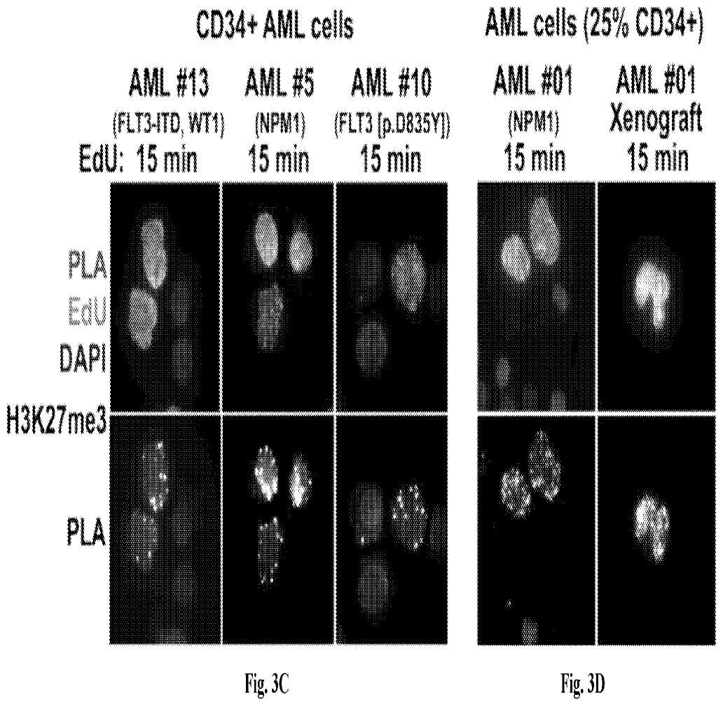

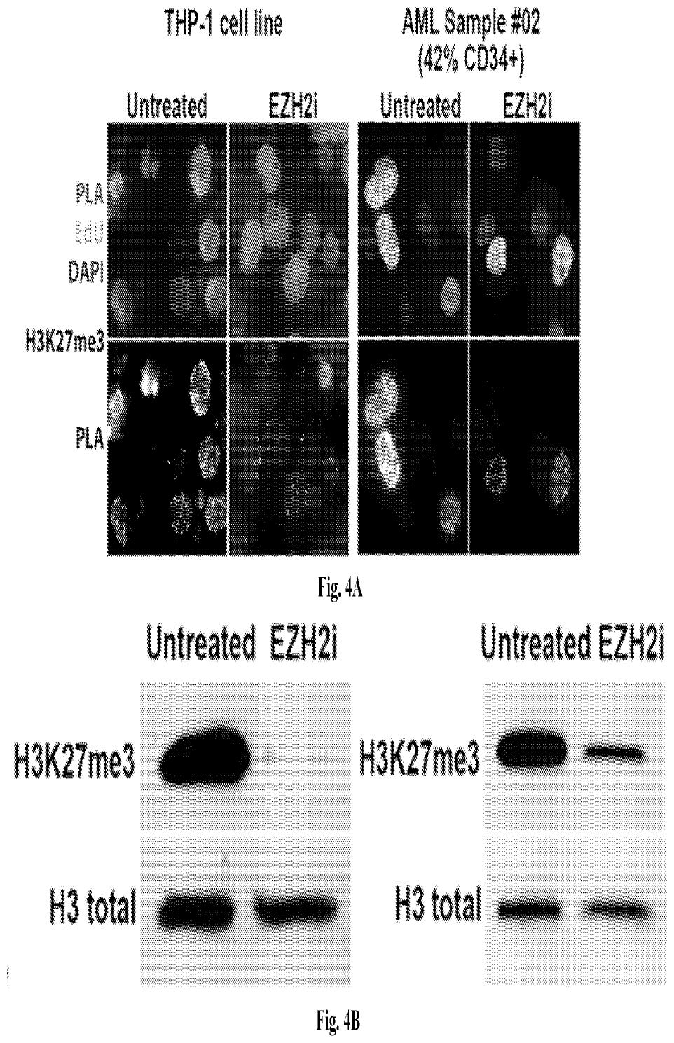

Cells of the THP-1 AML, cell line were stained using the CAA (FIG. 3A). Quantification (FIG. 3B) indicated that H3K27me3 accumulates very rapidly (and remains stably associated) on nascent DNA of AML cells, including in EdU-pulselabeled cells released from thymidine block. Similar results were observed in CD34+ cells from AML patients (examples in FIG. 4C), and blast cells from a mouse xenograft of primary AML (FIG. 3D). Rapid accumulation of H3K27me3 in AML cells appears to be independent of their FAB classification or the presence of specific genetic abnormalities. Importantly, absence of H3K27me3 correlates with a de-condensed structure of nucleosomes while the rapid accumulation of H3K27me3 on nascent DNA indicates that chromatin has acquired a “condensed” structure. In summary, normal HPCs have a de-condensed structure of nucleosomes on nascent DNA while CD34+ AML cells possess aberrant “condensed” post-replicative chromatin characterized by fast accumulation of the H3K27me3 repressive mark on nascent DNA. This feature may make AML cells less sensitive to DNA damaging agents and thus resistant to therapy.

### Example 4

### The Structure of Post-Replicative Chromatin can be Altered by ‘Epigenetic’ Inhibitors

Since DNA-damaging agents (eg., Doxorubicin) preferentially induce double strand breaks (DSBs) in more de-condensed regions of chromatin, some epigenetic inhibitors may render leukemic cells more susceptible to DNA damage. First, the structure of post-replicative chromatin was examined to determine whether it could be manipulated by inhibitors of two homologous enzymes, EZH1 and EZH2, that methylate H3K27. In several AML samples, EZH2 was present on nascent DNA, suggesting that inhibiting its activity may affect the extent to which nascent DNA is associated with the H3K27me3 repressive mark. Indeed, pharmacological inhibition of EZH2 activity in THP-1 cells or blasts from a primary AML sample with a “condensed” H3K27me3-modified nascent chromatin led to a marked decrease in global and nascent DNA-associated H3K27me3 levels (FIG. 4A) which was verified by western blot (FIG. 4B). Thus, the structure of post-replicative chromatin can be changed from condensed (H3K27me3-containing) to ‘open’ (H3K27me3-unmodified) in order to assess whether this change in chromatin conformation increases the sensitivity of DNA from AML cells to DNA damaging agents.

### Example 5

### Sensitivity of DNA of Leukemic Cells to DSBs Depends on the Structure of Chromatin After DNA Replication

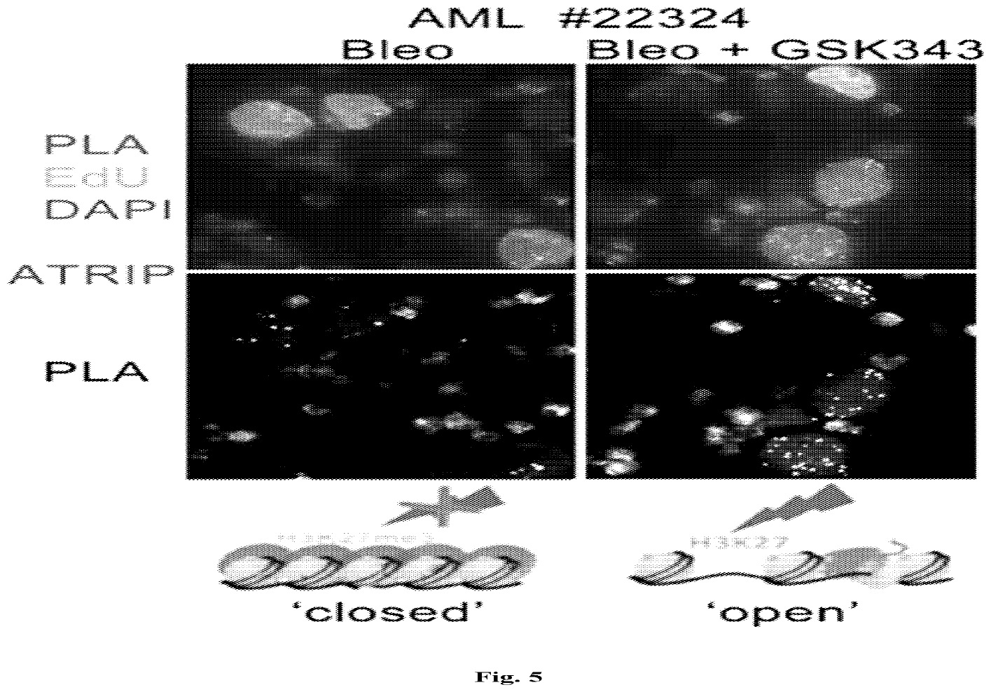

To increase the detection of DSBs, a new protocol was established based on the PLA detection of ATRIP or MDC1 (components of the ATR and ATM repair complexes, respectively) on EdU-labeled DNA at sites of DSBs. Advantages of this method are its high sensitivity and the absence of ATRIP and MDC1 on DNA prior to DNA damage (FIG. 5). Next, the sensitivity of primary leukemic cells to DNA damage following treatment to make post-replicative chromatin less condensed was assessed. Treatment of primary blasts from AML patient #22324 with the EZH2 inhibitor GSK343 to induced a post-replicative chromatin globally devoid of the H3K27me3 repressive mark significantly increased recruitment of ATRIP to sites of DNA damage induced by bleomycin (FIG. 5), suggesting that de-condensation of chromatin leads to a significant increase in bleomycin-induced DNA damage. These findings are potentially important for therapies combining DNA damaging agents and EZH1/EZH2 epigenetic inhibitors.

### Example 6

### Pharmacological Inhibition of EZH2 Enhances DNA Damage in AML Cells Treated with Cytotoxic Agents

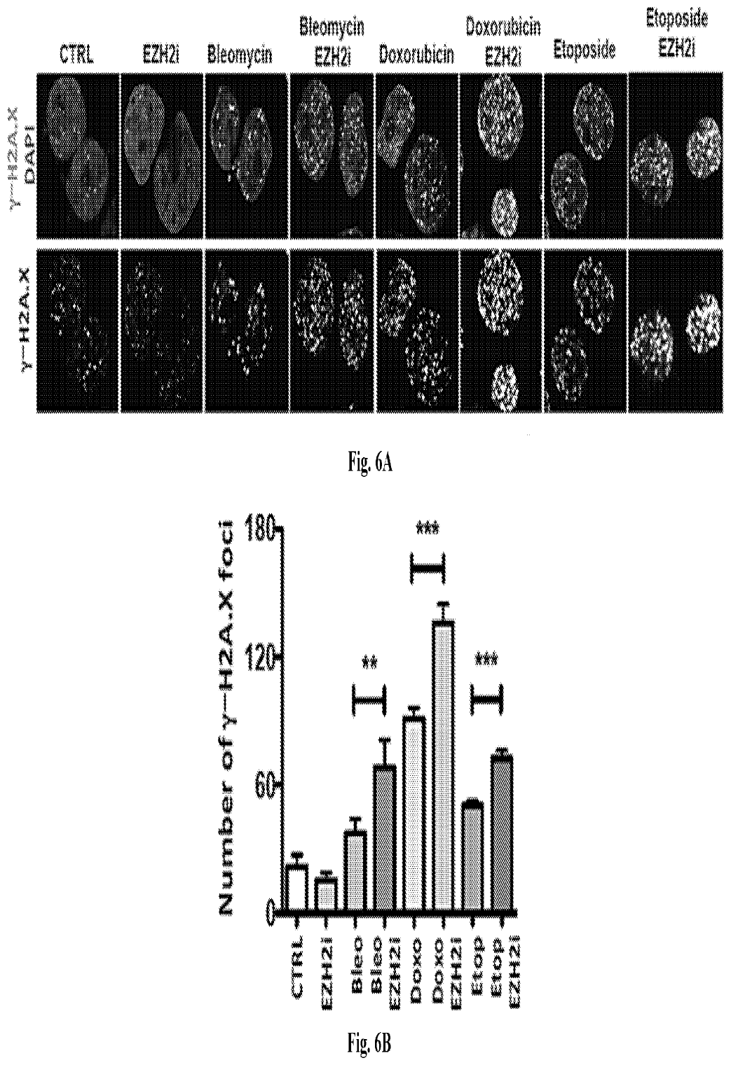

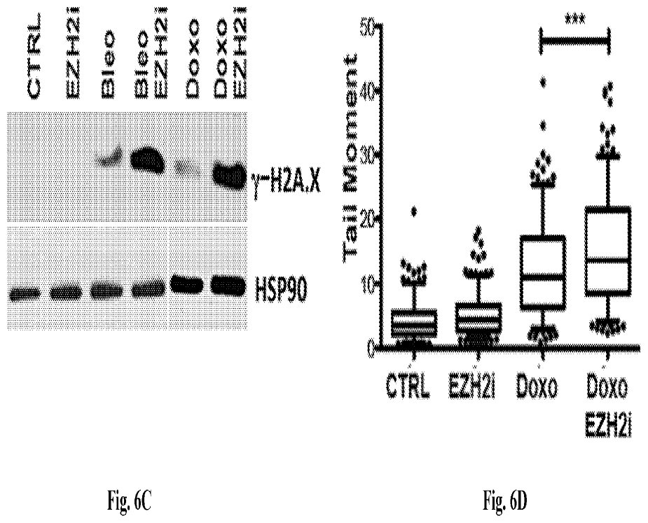

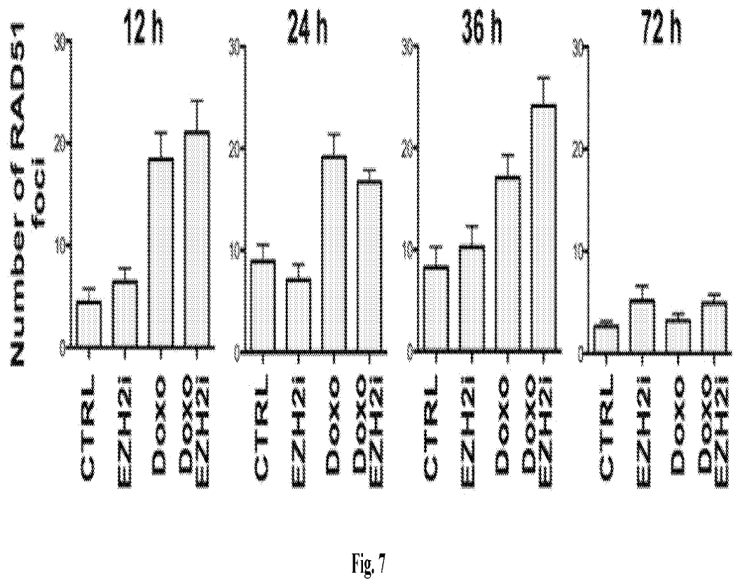

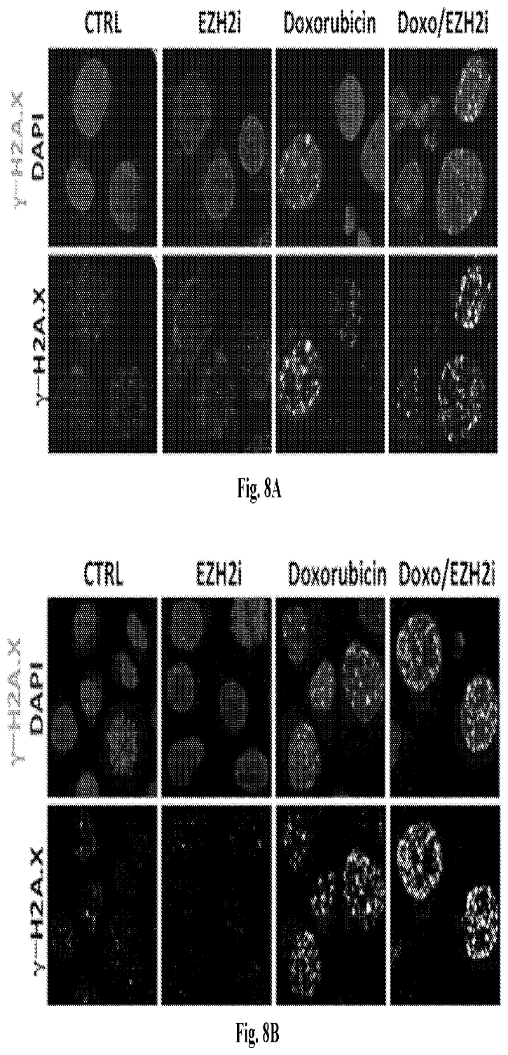

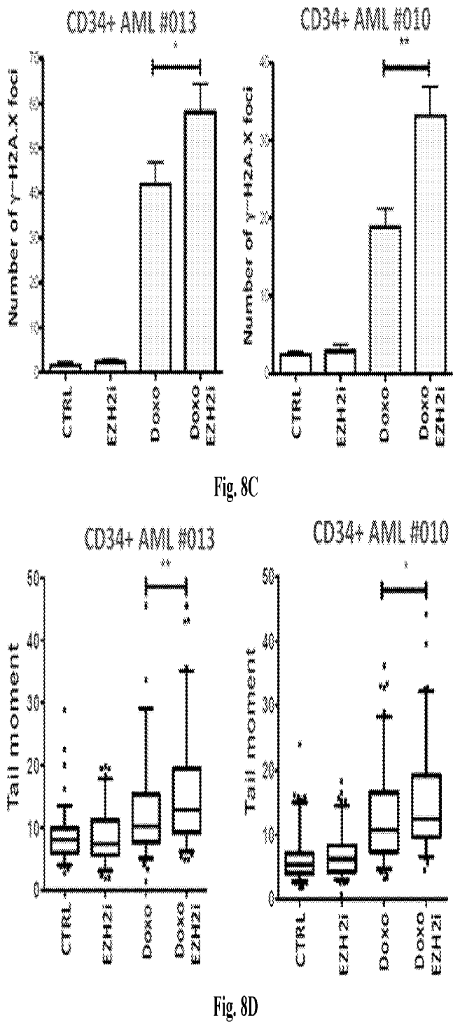

The role of chromatin structure in cytotoxic agent-induced DNA damage was also assessed by investigating the formation of γ-H2A.X foci by confocal microscopy. Treatment of THP-1 cells (expressing the MLL-AF9 chimeric protein and exhibiting very rapid accumulation of H3K27me3 on nascent DNA) with bleomycin, Doxorubicin or etoposide markedly increased the number of γ-H2A.X foci compared to untreated cells (FIG. 6A). Number and mean fluorescence intensity of foci, as well as γ-H2A.X levels, were further increased by co-treatment with GSK126 (FIGS. 6B and 6C). Increased DNA damage induced by co-treatment with GSK126 and Doxorubicin was independently confirmed by the comet assay (FIG. 6D). This increase is not due to defective DNA repair, since the number of RAD51 foci (indicative of DSBs repair) was not diminished by GSK126 treatment at any time point following Doxorubicin-induced DNA damage (FIG. 7). Higher levels of DNA damage, examined by analysis of γ-H2A.X foci and by the comet assay were also observed in CD34+ AML primary cells co-treated with GSK126 and Doxorubicin (FIGS. 8A-8D). As expected, treatment of THP-1 cells with the UTX/JMJD3 H3K27me3 demethylase inhibitor GSKJ4 to maintain the chromatin in a ‘condensed’ state did not enhance Doxorubicin-induced DNA damage.

### Example 7

### Co-Treatment with the EZH2 Inhibitor GSK126 and a Cytotoxic Agent Enhances Apoptosis of AML Cells

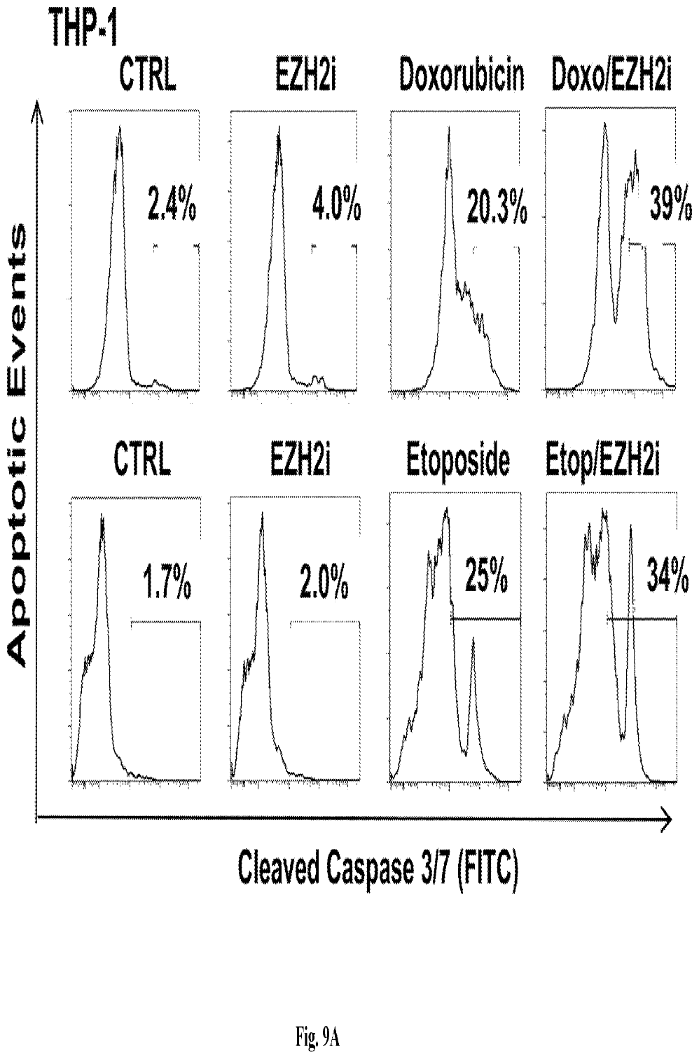

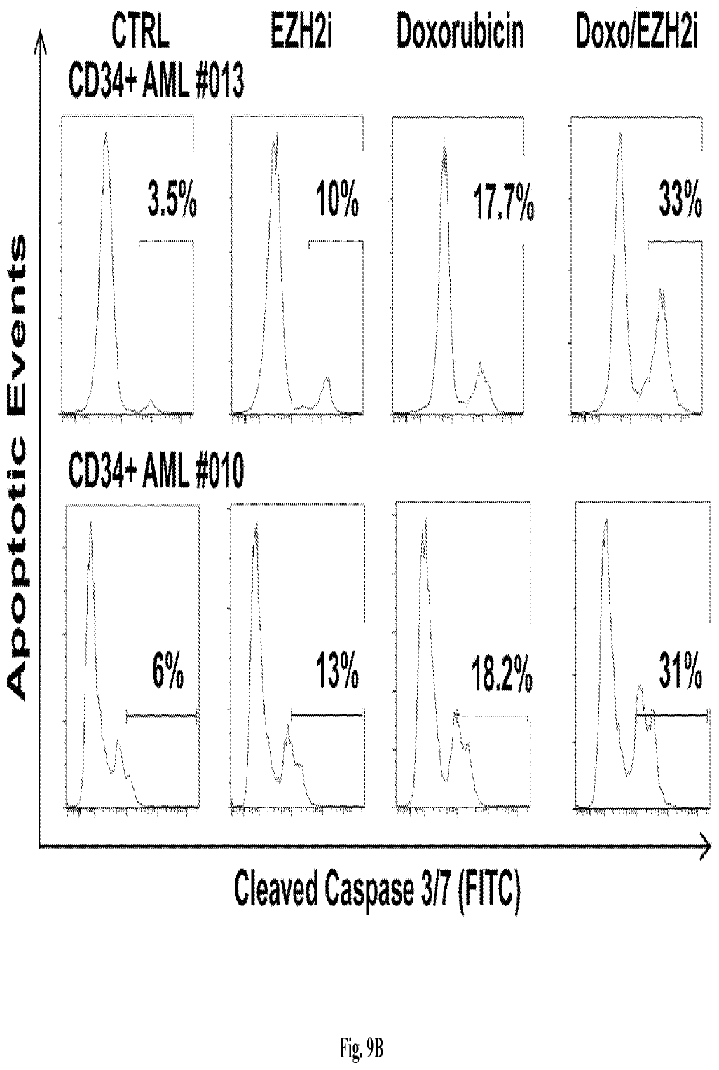

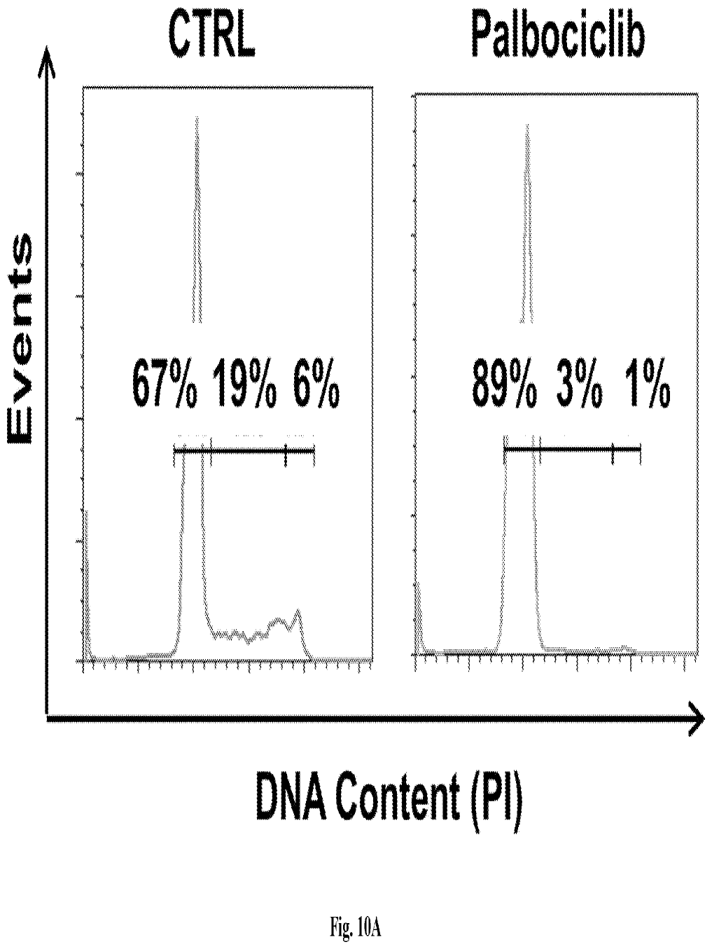

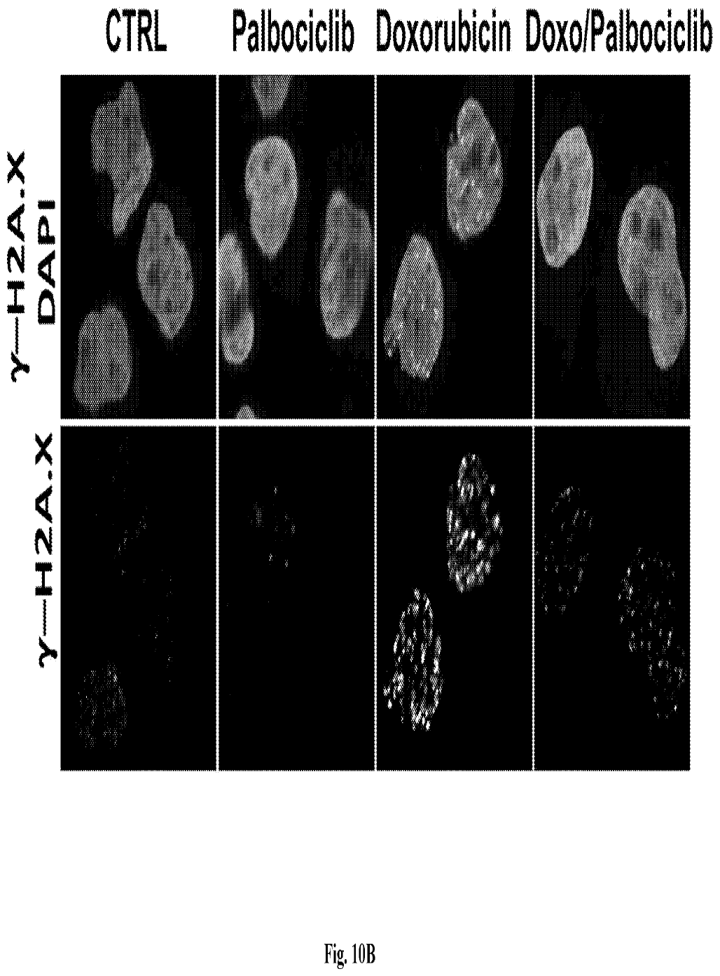

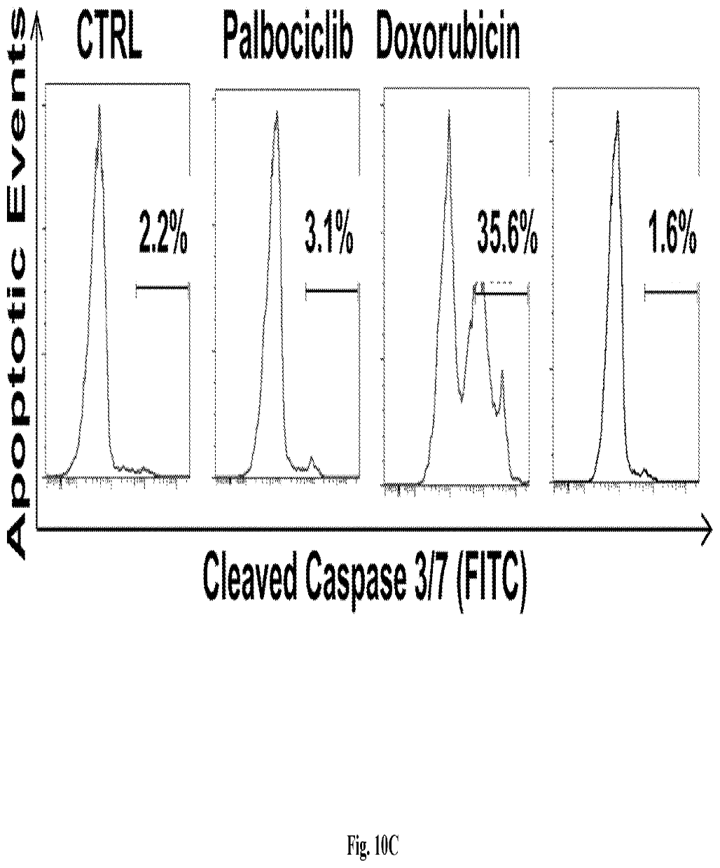

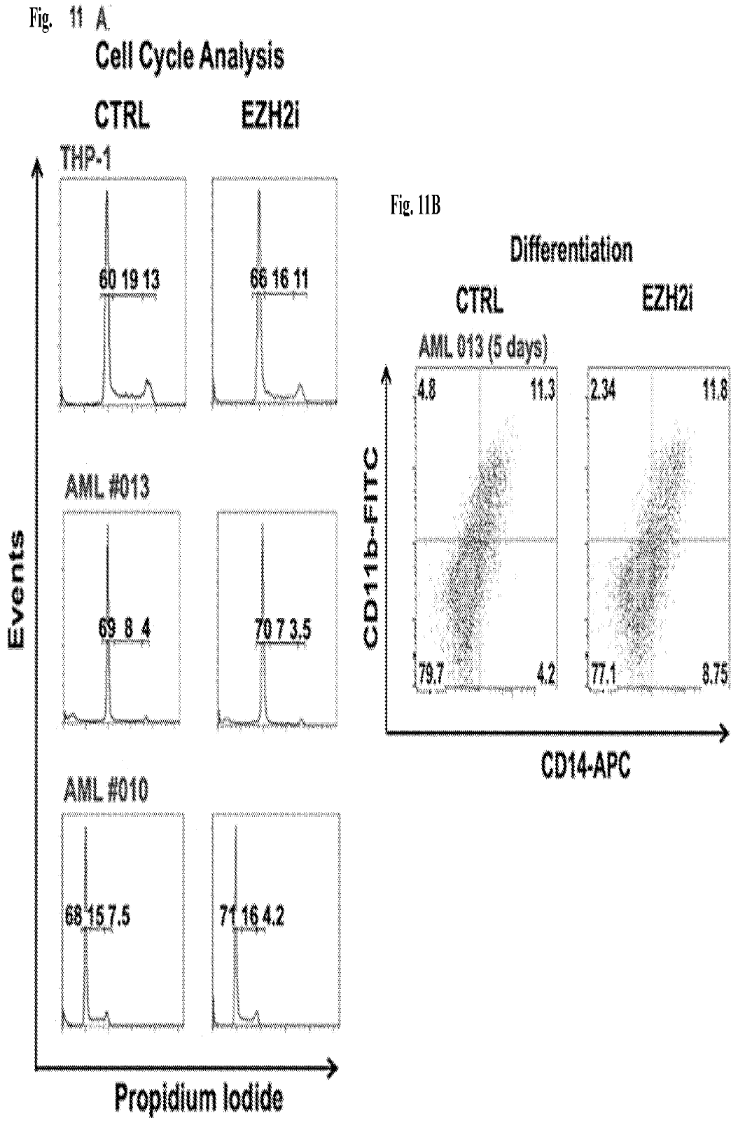

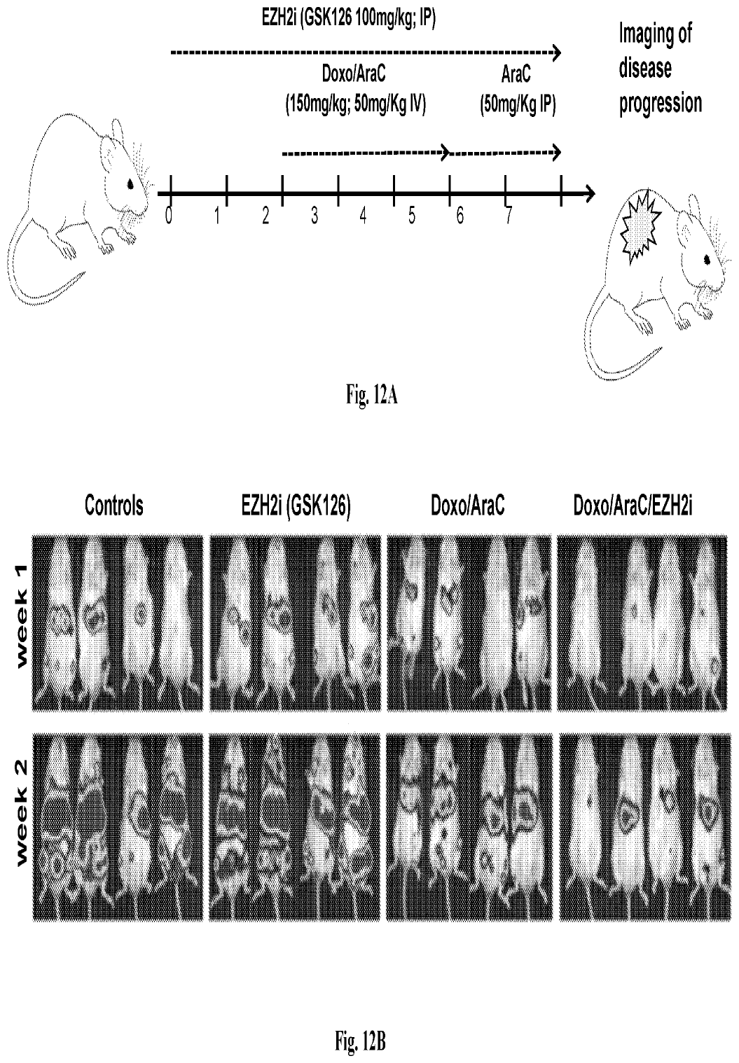

The increased DNA damage induced by co-treatment with GSK126 and Doxorubicin (FIGS. 6 and 8) correlated with enhanced apoptosis in THP-1 and CD34+ AML cells (FIGS. 9A and 9B). Similar results were obtained in AML cells co-treated with the EZH1/EZH2 inhibitor UNC1999 and bleomycin or Doxorubicin. Importantly, Doxorubicin-induced DNA damage and apoptosis and their potentiation by EZH2 inhibition are dependent on DNA synthesis since both were suppressed in THP-1 cells treated with the CDK4/6 inhibitor palbociclib (FIGS. 10B and 10C) which blocks the cells in G1 preventing S phase entry (FIG. 10A). Treatment with GSK126 alone had no effect on the proliferation (FIG. 11A) or the differentiation of AML cells (FIG. 11B), suggesting that enhanced DNA damage induced by EZH2 inhibition is independent from global effects of GSK126 on these two processes.

To assess whether cell cycle synchronization through pre-conditioning with a CDK4/6 inhibitor prior to treatment, a subsequent study was performed in which THP-1 cells were treated with Palbociclib (500 nM) for 24 hours to induce a G1-arrest. Palbociclib was then washed out and THP-1 cells re-entering S-phase were treated with GSK126 (5 μM) for 12 hours, before adding the Doxorubicin (0.1 μM); (FIG. 15, Left) DNA damage was assessed via the quantification of the number of g-H2A.X foci per nucleus. (FIG. 15, Right) Apoptosis of treated cells was also analyzed by Caspase 3/7 activity by flow cytometry. Together these results suggested that the apoptotic synergy observed with the combination of GSK126 and Doxorubicin treatment could be further enhanced by synchronizing the replication cycles of the cells via pre-treatment with a CDK 4/6 inhibitor.

### Example 8

### Co-Treatment with GSK126 Enhances the Effect of Doxorubicin/Ara-C in Suppressing AML Burden in Mice

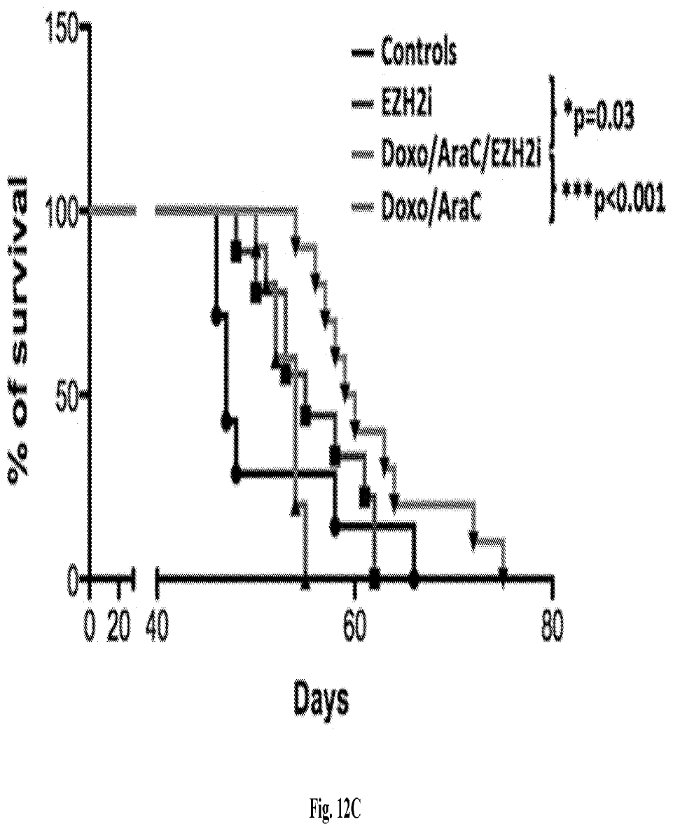

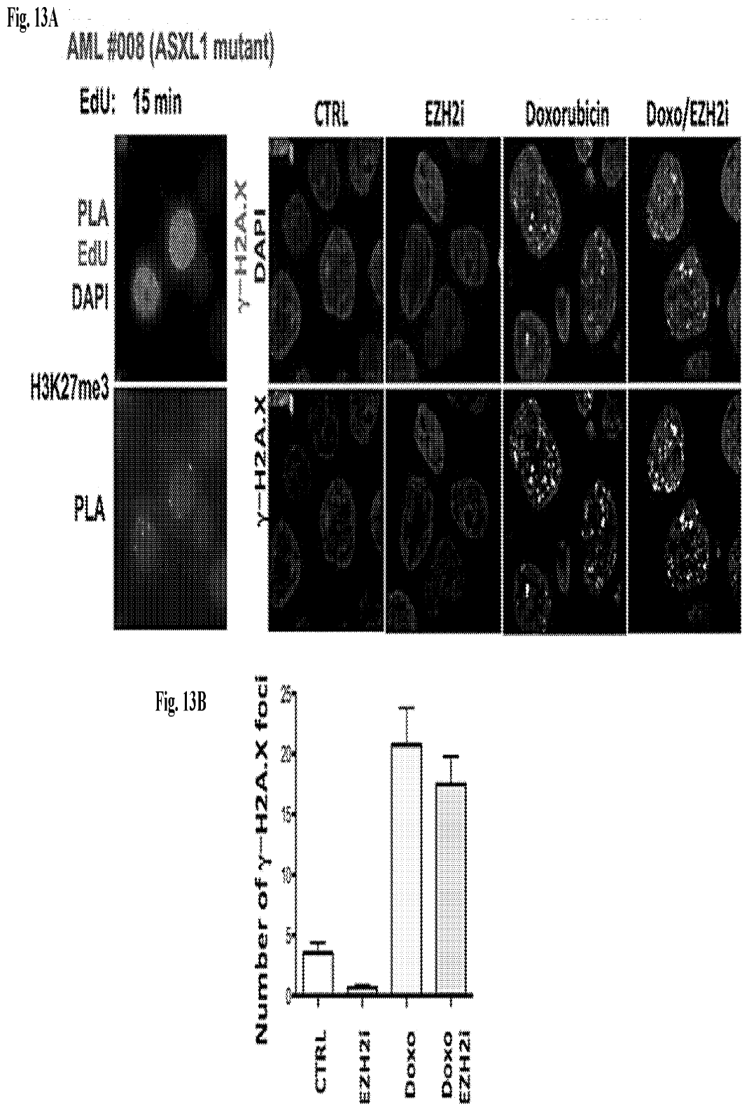

The potential of EZH2 pharmacological inhibition to enhance the anti-leukemia effect of cytotoxic agents inducing DNA damage was first investigated in NOD-Rag1null IL2rgnull (NRG) mice injected with the Luc-positive THP-1 AML cell line (FIG. 12A). The Doxorubicin/Ara-C combination was used because it is the induction therapy of choice for newly diagnosed AML; however, GSK126 may only potentiate the effects of Doxorubicin since ex vino studies in THP-1 cells indicate that it did not enhance DNA damage/apoptosis induced by treatment with Ara-C only. In this model of aggressive, MLL-AF9-driven leukemia the Doxorubicin/Ara-C and GSK126 combination appears to be more effective than Doxorubicin/Ara-C or GSK126 alone in suppressing leukemia burden (by bio-imaging) and in prolonging overall survival (FIGS. 12B and 12C). This is a significant finding since the THP-1 line mimics the behavior of AML PDX models that respond poorly to standard chemotherapy.

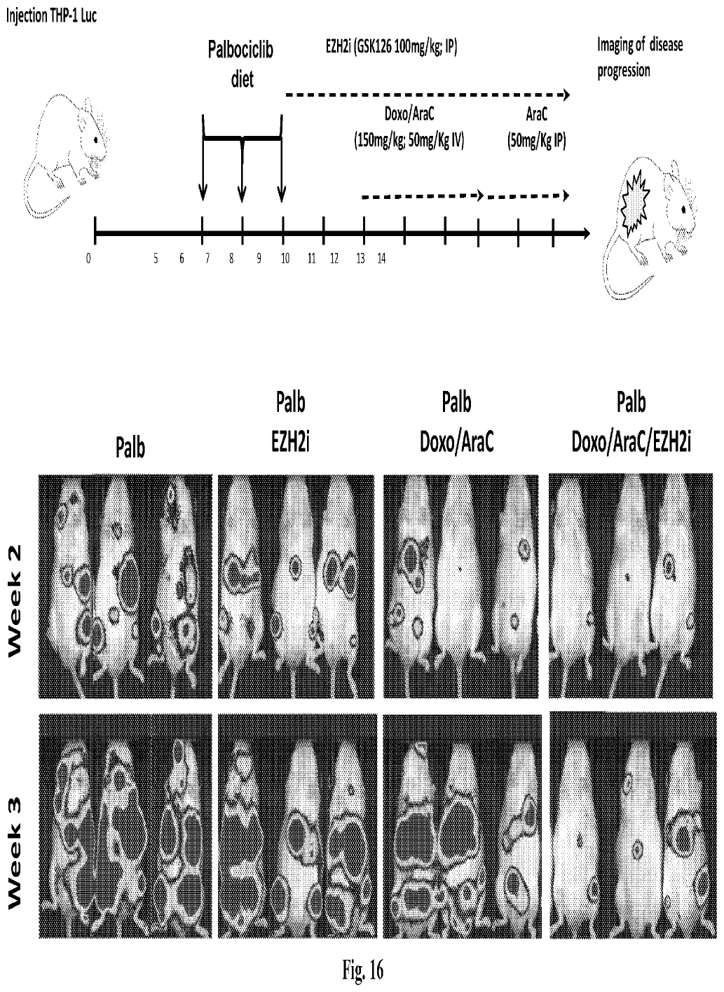

A subsequent study was then conducted in which treatment with GSK126 and Doxorubicin/Ara-C was combined with pre-treatment with the cell cycle inhibitor Palbociclib. (FIG. 16, Top) NRG mice were injected with THP-1-Luc+ cells (1×10⁶ cells/mouse). 5 days post-injection, mice were fed with chow containing 150 mg/kg of Palbociclib for 3 days. Then, mice were left untreated (Palb group), treated with GSK126 alone for 7 days (Palb/EZH2i group) or pre-treated (2 days) with GSK126 and the co-treated with GSK126 and chemotherapy for 5 additional days (Doxo/Ara-C/EZH2i group); the Palb/Doxo/Ara-C only group was treated for 5 days, starting 10 days after THP-1 cells injection (FIG. 16, Bottom) Serial bioluminescence images of mice were acquired 2 and 3 weeks after the end of the treatment. Without wishing to be bound by theory, these data suggested the utility of combination of the GSK126/Doxorubicin/Ara-C treatment regimen with pre-treatment with the cell cycle inhibitor Palbociclib.

### Example 9

### Effect of Mutations on H3K27me3 Accumulation

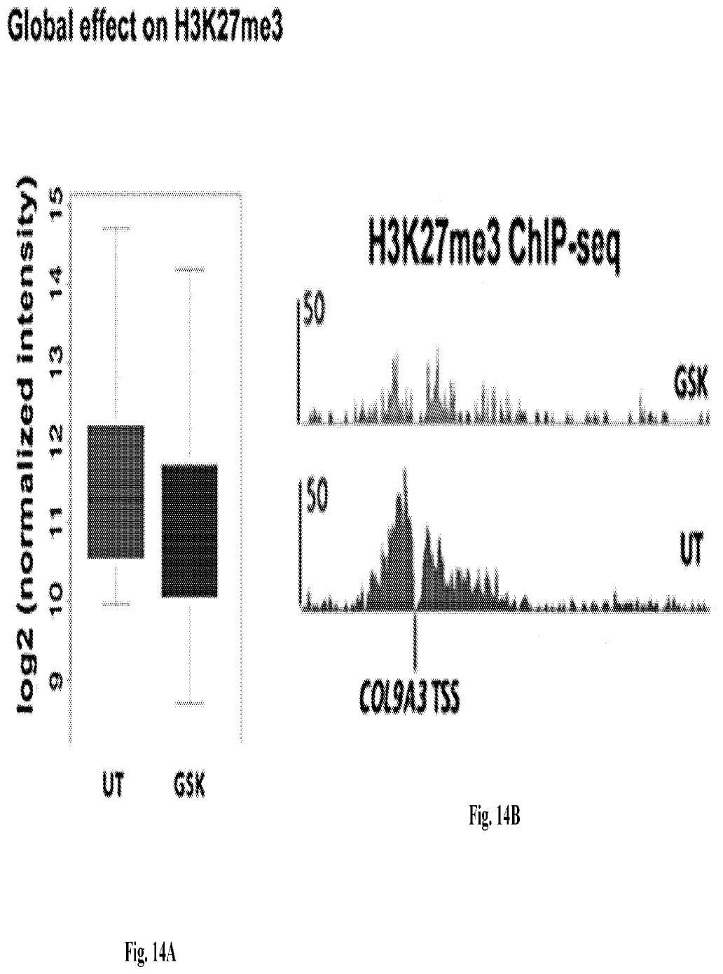

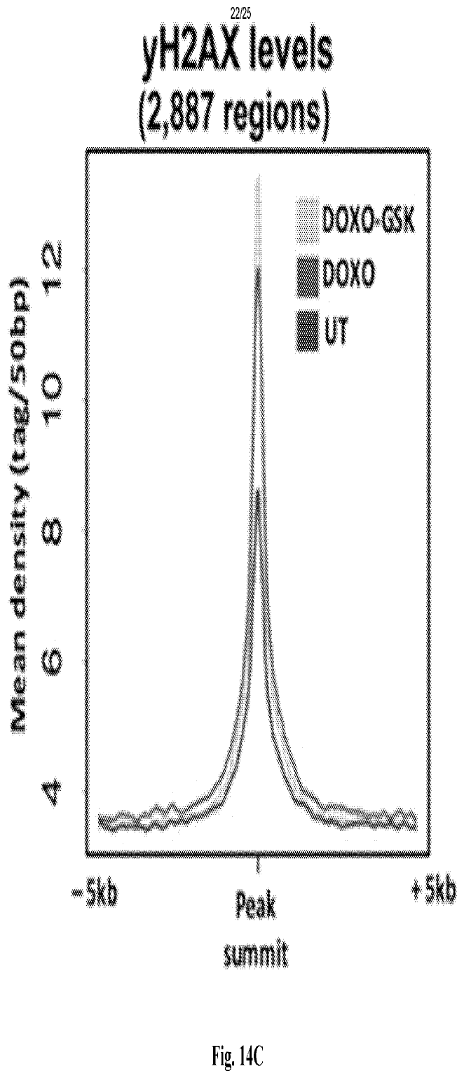

Mutations of ASXL1 are more frequent than EZH2 mutations and may cause a decrease in global H3K27me3 level and in the accumulation of this marker on nascent DNA. The H3K27me3 marker was not detected on Edu-labeled, nascent DNA of CD34+ XML cells carrying a frameshift ASXL1 mutation causing a premature termination (G646fs*12) in ˜50% of the cells, favoring a state of ‘open’ chromatin (FIGS. 13A and 13B). In these cells, co-treatment with GSK126 did not increase Doxorubicin-induced DNA damage.

### Example 10

### Effects of H3K27me3 Expression on the DNA Damage Response

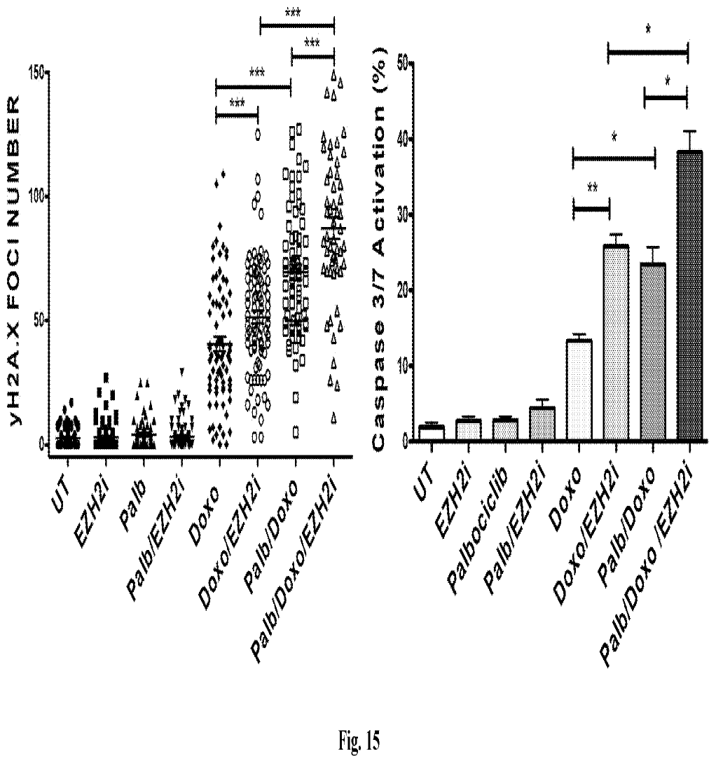

The effects of DNA damage were examined by performing ChIP-seq profiles of γ-H2A.X in THP-1 cells, untreated, Doxorubicin-treated, or co-treated with the EZH2 inhibitor GSK126 using a ChIP-seq experiment. Treatment of THP-1 cells with GSK126 induced a marked decrease in global levels of H3K27me3 (FIGS. 14A-B) and Doxorubicin-induced γ-H2A.X signals were increased in THP-1 cells co-treated with GSK126 (FIG. 14C). Without wishing to be bound by theory, a more relaxed chromatin structure may be more susceptible to DNA damage.

### Example 11

### Effect of the GSK126/Doxo/Ara-C Therapy in a Patient Derived Xenograft (PDX) Model of AML

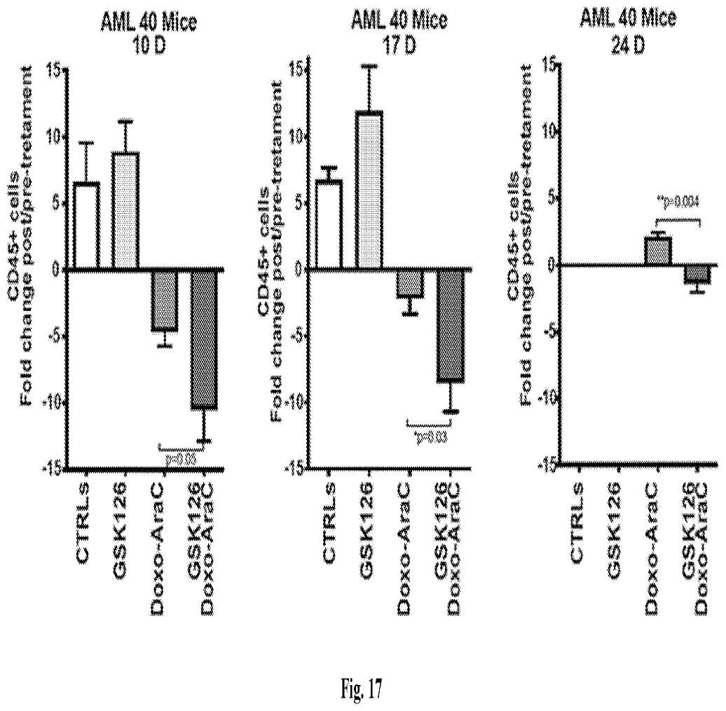

Because previous in vitro studies had utilized well-established cell lines, a series of studies was then undertaken to examine the effects of combination GSK126/Doxorubicin/Ara-C treatment on a xenograft mouse model using primary human tumor cells. Here, NOD-Rag1^{null} IL2rγ^{null} mice expressing human IL-3, human granulocyte/macrophage colony stimulating factor (GM-CSF), and human Steel factor (SF) (NRG-SGM3) were implanted with AML sample #040 cells (1.5×10⁶ cells/mouse). The human cytokines and factors expressed by NRG-SGM3 are especially suitable for engraftment of human leukemias. When human CD45-positive cells reached >10% in peripherals blood, mice were divided in 4 groups and treated with vehicle, GSK126, Doxo/Ara-C, or the Doxo/Ara-C/GSK126 combination. Leukemia burden was then assessed by measuring the % of human CD45⁺ leukemic cells in the peripheral blood at one-week intervals starting 10 days after therapy cessation (FIG. 17). The GSK126/chemotherapy combination was observed to be more effective than treatment with Doxo/Ara-C alone. For these studies, both Doxo and Ara-C were given at a lower dose (Doxo/Ara-C 5+3: 1.0 mg/Kg/33.3 mg/Kg, which is one-third lower than what used in the THP-1 model in FIG. 8). The effect of drug treatment on leukemia burden was monitored by assessing the percentage of CD45+ leukemic cells three times at one-week intervals starting 10 days after termination of the therapy.

Vehicle-treated and GSK126-treated mice exhibited similar increases in leukemia burden over the course of the experiment. Treatment with Doxo/Ara-C only suppressed AML cell growth; however, the effect was transient because the percentage of peripheral blood leukemic cells 24 days after therapy cessation was higher than at the start of the treatment. By contrast, the combined GSK126/Doxo/Ara-C treatment was clearly more effective than chemotherapy alone, exhibiting a significant improvement in AML growth suppression over treatment with chemotherapy alone at each time point of analysis (FIG. 17). These results demonstrated the clinical utility of this triple-combination for the treatment of leukemias, including AML.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating cancer in a subject, the method comprising administering to the subject:
an effective amount of a histone methyltransferase inhibitor, and
an effective amount of a chemotherapy.

2. The method of claim 1, further comprising administering to the subject an effective amount of a cell cycle inhibitor.

3. The method of claim 2, wherein the cell cycle inhibitor is administered first, followed by the histone methyltransferase inhibitor, followed by the chemotherapy.

4. The method of claim 2, wherein the cell cycle inhibitor, histone methyltransferase inhibitor, and chemotherapy are administered concurrently.

5. The method of claim 2, wherein the cell cycle inhibitor is administered first, followed by concurrent administration of the histone methyltransferase inhibitor and chemotherapy.

6. The method of claim 2, wherein the cell cycle inhibitor is an inhibitor of any one or both of CDK4 and CDK6.

7. The method of claim 6, wherein the CDK4 and CDK6 inhibitor is selected from the group consisting of abemaciclib, palbociclib, and ribociclib.

8. The method of claim 1, wherein the histone methyltransferase inhibitor is an inhibitor of the methylation of H3K27.

9. The method of claim 8, wherein the histone methyltransferase inhibitor is an inhibitor of enzyme Enhancer of Zeste Homolog 2 (EZH2).

10. The method of claim 9, wherein the EZH2 inhibitor is selected from the group consisting of tazemetostat, GSK126, 3-deazaneplanocin A, and GSK343.

11. The method of claim 1, wherein the chemotherapy is a DNA-damaging agent.

12. The method of claim 11, wherein the DNA-damaging agent is selected from the group consisting of a platinum drug and a Topoisomerase II inhibitor drug.

13. The method of claim 12, wherein the platinum drug is selected from the group consisting of cisplatin, carboplatin, oxaliplatin, and nedaplatin.

14. The method of claim 12, wherein the Topoisomerase II inhibitor is an anthracycline drug selected from the group consisting of Doxorubicin, daunorubicin, epirubicin, and mitoxantrone.

15. The method of claim 12, wherein the Topoisomerase II inhibitor is etoposide.

16. The method of claim 1, wherein the chemotherapy is bleomycin.

17. The method of claim 1, wherein the chemotherapy is an antimetabolite.

18. The method of claim 17, wherein the antimetabolite is selected from the group consisting of cytarabine, mercaptopurine, fludarabine, hydroxycarbamide, and methotrexate.

19. The method of claim 1, further comprising administering to the subject an effective amount of one or more additional chemotherapies.

20. The method of claim 19, wherein the one or more additional chemotherapies are selected from the group consisting of a platinum drug, a Topoisomerase II inhibitor drug, an anthracycline drug, an antimetabolite drug, bleomycin, cisplatin, carboplatin, oxaliplatin, nedaplatin, Doxorubicin, daunorubicin, epirubicin, mitoxantrone, cytarabine, mercaptopurine, fludarabine, hydroxycarbamide, and methotrexate.

21. A method of treating cancer in a subject, the method comprising administering to the subject an effective amount of GSK126, an effective amount of Doxorubicin, and an effective amount of cytarabine.

22. The method of claim 21, wherein the subject is pre-screened for an ASXL1 mutation, wherein when the subject carries the ASXL1 mutation, an alternative treatment is administered.

23. The method of claim 21, further comprising administering to the subject an effective amount of one or more additional chemotherapies.

24. The method of claim 23, wherein the one or more additional chemotherapies are selected from the group consisting of a platinum drug, a Topoisomerase II inhibitor drug, an anthracycline drug, an antimetabolite drug, bleomycin, cisplatin, carboplatin, oxaliplatin, nedaplatin, Doxorubicin, daunorubicin, epirubicin, mitoxantrone, cytarabine, mercaptopurine, fludarabine, hydroxycarbamide, and methotrexate.

25. The method of claim 1, wherein the subject is a human.

26. The method of claim 1, wherein the cancer is a hematologic malignancy.

27. The method of claim 26, wherein the hematologic malignancy is selected from the group consisting of acute myelogenous leukemia, acute myeloblastic leukemia, acute myeloid leukemia, and acute nonlymphocytic leukemia, acute monocytic leukemia, acute monoblastic leukemia, acute megakaryocytic leukemia, acute erythroblastic leukemia, and chronic myelogenous leukemia-blast crisis.

28. A method of treating cancer in a subject, comprising:
a) administering to a sample from the subject, a plurality of combinations of histone methyltransferase inhibitors and chemotherapies,
b) determining the optimal combination of histone methyltransferase inhibitors and chemotherapies, and
c) treating the subject with the optimal combination of histone methyltransferase inhibitors and chemotherapies.

29. The method of claim 28, wherein the histone methyltransferase inhibitor is an inhibitor of the methylation of H3K27.

30. The method of claim 28, wherein the histone methyltransferase inhibitor is an inhibitor of enzyme Enhancer of Zeste Homolog 2 (EZH2).

31. The method of claim 30, wherein the EZH2 inhibitor is selected from the group consisting of tazemetostat, GSK126, 3-deazaneplanocin A, and GSK343.

32. The method of claim 28, wherein the chemotherapy is a DNA-damaging agent.

33. The method of claim 32, wherein the DNA-damaging agent is selected from the group consisting of a platinum drug and a Topoisomerase II inhibitor drug.

34. The method of claim 33, wherein the platinum drug is selected from the group consisting of cisplatin, carboplatin, oxaliplatin, and nedaplatin.

35. The method of claim 33, wherein the Topoisomerase II inhibitor is an anthracycline drug selected from the group consisting of Doxorubicin, daunorubicin, epirubicin, and mitoxantrone.

36. The method of claim 33, wherein the Topoisomerase II inhibitor is etoposide.

37. The method of claim 27, wherein the chemotherapy is bleomycin.

38. The method of claim 28, wherein the chemotherapy is an antimetabolite.

39. The method of claim 38, wherein the antimetabolite is selected from the group consisting of cytarabine, mercaptopurine, fludarabine, hydroxycarbamide, and methotrexate.

40. The method of claim 28, further comprising administering to the subject an effective amount of one or more additional chemotherapies.

41. The method of claim 40, wherein the one or more additional chemotherapies are selected from the group consisting of a platinum drug, a Topoisomerase II inhibitor drug, an anthracycline drug, an antimetabolite drug, bleomycin, cisplatin, carboplatin, oxaliplatin, nedaplatin, Doxorubicin, daunorubicin, epirubicin, mitoxantrone, cytarabine, mercaptopurine, fludarabine, hydroxycarbamide, and methotrexate.

42. The method of claim 28, wherein the subject is a human.

43. The method of claim 28, wherein the cancer is a hematologic malignancy.

44. The method of claim 43, wherein the hematologic malignancy is selected from the group consisting of acute myelogenous leukemia, acute myeloblastic leukemia, acute myeloid leukemia, and acute nonlymphocytic leukemia, acute monocytic leukemia, acute monoblastic leukemia, acute megakaryocytic leukemia, acute erythroblastic leukemia, and chronic myelogenous leukemia-blast crisis.
